(19) United States
(12) Patent Application Publication
(10) Pub. No.: US 2004/0002090 A1
(43) Pub. Date: Jan. 1, 2004

(54) Methods for detecting genome-wide sequence variations associated with a phenotype

(76) Inventors: Pascal Mayer, Eloise (FR); Ilia Leviev, Apples (CH); Magne Osteras, Gland (CH); Laurent Farinelli, Meyrin (CH)

Correspondence Address: PENNIE AND EDMONDS, 1155 AVENUE OF THE AMERICAS, NEW YORK, NY, 100362711

(21) Appl. No.: 10/378,688

(22) Filed: Mar. 4, 2003

Related U.S. Application Data
(60) Provisional application No. 60/362,023, filed on Mar. 5, 2002.

(51) Int. Cl. C12Q 1/68; C12P 19/34

(52) U.S. Cl. 435/6; 435/91.2

(57) ABSTRACT

The invention provides methods for determining genome-wide sequence variations associated with a phenotype of a species in a hypothesis-free manner. In the methods of the invention, a set of restriction fragments for each of a sub-population of individuals having the phenotype are generated by digesting nucleic acids from the individual using one or more different restriction enzymes. A set of restriction sequence tags for the individual is then determined from the set of restriction fragments. The restriction sequence tags for the sub-population of organisms are compared and grouped into one or more groups, each of which comprising restriction sequence tags that comprise homologous sequences. The obtained one or more groups of restriction sequence tags identify the sequence variations associated with the phenotype. The methods of the invention can be used for, e.g., analysis of large numbers of sequence variants in many patient samples to identify subtle genetic risk factors.

This application claims benefit, under 35 U.S.C. §119(e), of U.S. Provisional Patent Application No. 60/362,023, filed on Mar. 5, 2002, which is incorporated herein by reference in its entirety.

### 1. FIELD OF THE INVENTION

The present invention relates to methods for detecting in a population of organisms of a species genome-wide sequence variations associated with a phenotype in a hypothesis-free manner. The present invention also relates to methods for generating genome-wide restriction sequence tags for an organism.

### 2. BACKGROUND OF THE INVENTION

Molecular approaches for genetic analyses trace the nucleotide sequence variations that occur naturally and randomly in the genomes of organisms. Knowledge of DNA polymorphisms among individuals and between populations is important in understanding the complex links between genotypic and phenotypic variations. In the absence of complete data about sequence variation, one relies on the ability to identify ‘nearby’ markers that allow one to infer the location of certain relevant loci or causal sequence variations. The informativeness of the markers depends on the magnitude of the linkage disequilibrium. Markers can be used in linkage studies to search for candidate genes and in association studies to identify the functional allelic variations of candidate genes that influence inter-individual variations.

In order to link adverse response to drug treatment and susceptibility to diseases to the genomic makeups of individuals, it is necessary to monitor the differences in the genome among individuals. The current approach includes monitoring a large set of genetic markers, e.g., thousands of Single Nucleotide Polymorphisms (SNPs) evenly spread over the genome. These SNPs are monitored in individuals from a control population and in individuals in an affected population, or more generally, a population with a given phenotype. Linkage disequilibrium between the two populations for given SNPs is then used as an indication for the physical proximity on the genome between the SNPs and genomic regions involved in the drug response or disease susceptibility.

SNPs are the most common form of genetic polymorphism. This coupled with their potential as functional variants, has produced a great deal of interest in SNPs both as pharmacogenetic indicators and as markers for mapping genes for mapping genes for complex diseases (Risch et al., 1996, Science 273:1516-7; Kruglyak, 1997, Nat. Genet. 17:21-4; Masood, 1999, Nature 398:545-6). A large number of SNPs have already been identified with >2,500,000 entries on the NCBI's SNP database alone (http://www.ncbi.nlm.nih.gov/SNP/). Many recent studies are focused on identifying polymorphisms that lie in the coding sequence of potential candidate genes associated with common diseases (Nickerson et al., 1998, Nat. Genet. 19:233-240; Cambien et al., 1999, Am. J. Hum. Genet. 65:183-91; Risch et al., 1996, Science 273:1516-7; Kruglyak, 1997, Nat. Genet. 17:21-4; Masood, 1999, Nature 398:545-6; Cargill et al., 1999, Nat. Genet. 22:231-238; Halushka et al., 1999, Nat. Genet. 22:239-247).

In the present state of the art, the SNPs have to be first discovered by intensive resequencing of large portions of the genome of individuals belonging to a well chosen control population on the order of 100 individuals. The most common differences found are candidates for SNPs. This approach is very time consuming and expensive and the result is dependent on the choice of the control population.

Once the SNPs are identified, methods have to be developed that allow for the fast and cost effective scoring of a large number of SNPs in an individual. In the present state of the art, most methods used to score an SNP rely on the amplification by PCR (or alternative DNA amplification methods) of a small region surrounding the SNP. This amplification step requires the knowledge of the sequence surrounding the SNP and the use of specific and custom made nucleic acid primers for each SNP. Simultaneous amplification of a large number of different DNA sequences is a tedious and expensive process, requiring sophisticated and expensive robotics and a large amount of expensive reactants.

The ability to genotype this abundant source of variation rapidly and accurately is becoming an ever more important goal in the genetics community (Bonn, D., 1999, Lancet, 353:1684). A variety of technologies available have the potential to transfer to high-throughput genotyping laboratories (Landegren et al., 1998, Genome Research 8:769-776). These include 5′ exonuclease assays, such as TaqMan (Lyvak et al.,1995, Nature Genet. 9:341-342), molecular beacons (Tyagi et al, 1998, Nat. Biotechnol. 16:49-53), oligonucleotide-ligation assays (OLAs) (Tobe et al., 1996, Nucleic Acids Res. 24:3728-3732), dye-labeled oligonucleotide ligation (DOL) (Chen et al., 1998, Genome Res., 8:549-556), minisequencing (Chen et al., 1997, Nucleic Acids res., 25:347-353; Pastinen et al., 1997, Genome Res. 7:606-614), microarray technology (Hacia et al., 1998, Genome Res. 8:1245-1258; Wang et al., 1998, Science, 280:1077-1082) and the scorpions assay (Whitcombe et al., 1999, Nat. Biotechnol. 17:804-807).

These existing methods have two main bottlenecks: the first is that SNPs have to be identified and arbitrarily selected prior to scoring, and the second is that a large number of different DNA products have to generate by specific amplification. We therefore design a method that specifically avoids these two bottlenecks.

In the present state of the art, existing methods do not satisfy the needs of the pharmaceutical industry. Besides the pharmaceutical industry, many other fields such as medical research, healthcare management, veterinary, agricultural, food, cosmetics and many other industries and fields are interested in using the same approach based on different contexts and/or different organisms. A new method is thus needed for gaining full access to the abundant genetic variation of organisms at low cost, very high throughput and high accuracy.

Thus, there is a need for more efficient methods for analysis of large numbers of sequence variants in many patient samples to identify subtle genetic risk factors that go undetected in current genome scans by use of fewer markers, limited sample sizes, and/or pooled samples. It is therefore an object of the present invention to provide a more efficient method of detection of nucleic acid variation. It is also an object of the present invention to provide a more efficient method of sequencing.

Discussion or citation of a reference herein shall not be construed as an admission that such reference is prior art to the present invention.

### 3. SUMMARY OF THE INVENTION

The invention provides methods for determining genome-wide sequence variations associated with a phenotype of a species, preferably in a hypothesis-free manner. In one embodiment, the genome-wide variations are determined from a sub-population of individuals of a particular phenotype. In the methods of the invention, a set of restriction fragments for each individual in the sub-population of individuals having the phenotype are generated by digesting nucleic acids from the individual using one or more different restriction enzymes. Preferably, the set of restriction fragments comprises a sufficient number of different restriction fragments to permit identifying sequence variations in the genome of the organism. More preferably, the set of restriction fragments comprises a least 10, 100, 1000, 10⁴, 10⁵, 10⁶, 10⁷, or 10⁸ different restriction fragments.

A set of restriction sequence tags is then determined for each of the individuals from the set of restriction fragments of the individual. In the methods of the invention, a set of restriction sequence tags for an individual in the sub-population having the particular phenotype is preferably determined by generating a set of restriction fragments from, e.g., the genomic DNA, of the individual followed by sequencing a portion of each of the restriction fragments using a method comprising generation of DNA colonies (described infra).

The sets of restriction sequence tags obtained for different individuals in the sub-population are then preferably compared and grouped into one or more groups, each of which comprising restriction sequence tags that comprise homologous sequences. The comparison preferably permits determination of the number or frequency of each group of restriction sequence tag. The collection of the groups of homologous restriction tags for a sub-population can be used to identify sequence variations associated with the phenotype. In a preferred embodiment, the restriction sequence tags are compared with the genomic sequence of the organism to identify the genomic locations of the restriction sequence tags. In another preferred embodiment, the restriction sequence tags flanking both sides of the recognition sites are also identified from the genomic sequence of the organism.

The invention also provides methods for determining genome-wide sequence variations among a plurality of phenotypes by comparing the restriction sequence tags of different phenotypes. The methods of the invention are applicable to any species of organism. The methods of the invention are particularly useful for higher eukaryotic organisms which have complex genomes, such as higher animals, including but not limited to mammals including mice and preferably humans, and plants. In particular, the methods of the invention are useful for analysing and identifying sequence variations associated with disease susceptibility or response to treatments in humans.

### 4. BRIEF DESCRIPTION OF FIGURES

FIG. 1 illustrates a method for identification of restriction sequence tags associated with a phenotype.

FIGS. 2A and 2B illustrate an embodiment of the invention for the determination of restriction sequence tags.

FIGS. 3A and 3B illustrate an embodiment for the determination of restriction sequence tags by generating restriction fragments from the genome of an organism using a restriction enzyme that cuts on both sides of its recognition site.

FIGS. 4A and 4B illustrate an embodiment for the determination of restriction sequence tags by generating restriction fragments from the genome of an organism using a type IIs endonuclease.

FIGS. 5A and 5B illustrate an embodiment for the determination of restriction sequence tags by generating restriction fragments from the genome of an organism using double digestion: a rare cutter followed by a frequent cutter.

FIGS. 6A and 6B illustrate another embodiment for the determination of restriction sequence tags by generating restriction fragments from the genome of an organism using double digestion: a first restriction enzyme and a plurality of second restriction enzymes.

FIGS. 7A and 7B illustrate another embodiment for the determination of restriction sequence tags using by generating restriction fragments from the genome of an organism using double digestion: a first restriction enzyme and a plurality of second restriction enzymes.

FIGS. 8A and 8B illustrate another embodiment for the determination of restriction sequence tags using by generating restriction fragments from the genome of an organism using double digestion: a first restriction enzyme and a plurality of second restriction enzymes.

FIG. 9A illustrates the generation of short DNA tags from cloned DNA fragments. Long DNA fragments are cloned into circular vectors between two BsmFI sites. BsmFI digestion leaves only short DNA tags attached to the vector. After the self-ligation the circular vector contains an insert which is formed by the pair of tags regardless of the length of the original DNA fragment insert. FIG. 9B shows the results of an analysis of products after the first ligation. The Sau3AI digested lambda phage DNA was ligated with BamHI digested/dephophorylated 1^{st} generation vector. For analysis, the product were amplified by PCR using primer flanking the insertion site. FIG. 9C shows the results of an analysis of products after the second ligation. The same samples as in FIG. 9B were further processed to normalize their size by BsmFI digestion and self-ligation to generate the circular vector. For analysis, this product was amplified by PCR. The expected peak of 132 bp was observed. FIG. 9D shows the results of an analysis of the second ligation products obtained in a simplified reaction. A plasmid containing a single insert was treated with BsmFI and self-ligated after Klenow enzyme treatment to generate blunt ends. For analysis, the products were amplified by PCR. No bands corresponding to fragments of a size smaller than the correct size were observed.

FIG. 10A shows several possibilities of cloning of DNA generated by digestion using two different enzymes into a 2^{nd} generation vector. FIG. 10B shows the results of an analysis of in-vitro cloning into the 2^{nd} generation vector. MspI and SphI digested lambda DNA was inserted into a vector digested with SphI and AccI. After digestion with BsmFI, the second ligation resulted in normalized size inserts, the restriction sequence tags. The PCR products obtained by amplification of the final ligation reaction were analyzed. Only the band of correct size was observed. FIG. 10C shows the results of an analysis of products of a first ligation when AluI and SphI digested lambda DNA was inserted into a HincI and SphI digested vector. After PCR amplification for analysis, as expected fragments of different sizes were observed using Agilent 2100 bioanalyzer DNA 1000 chip for analysis. The highest peaks are the size markers. FIG. 10D shows the results of an analysis of the same samples as in FIG. 10C after the second ligation. After PCR amplification for analysis, only a single fragment of the expected size was observed using Agilent 2100 bioanalyzer DNA 1000 chip. Peaks corresponding to size markers are indicated in the figure.

FIGS. 11A-B illustrate the template preparation for HindIII and RsaI digested DNA using the single restriction sequence tag procedure illustrated on FIG. 4A. FIG. 11C shows aliquots collected after the various steps of the process and analysed by autoradiography. Lane 1: PCR product of complete DNA colony vector size, 350 bp; lane 2-6: lambda genomic DNA and lane 7-10 human genomic DNA; lane 3 and 7 after ligation to the short arm: multiple fragments or smear are observed; lane 4 and 8 after digest with MmeI, the size standardization is observed; lane 5, 6, 9 and 10: after ligation with the long arm thus generating the DNA colony vector with expected size. FIG. 11D shows DNA colonies of Lambda DNA. FIG. 11E shows DNA colonies of Lambda DNA (left column) or Human DNA (first 3 images of right column). These DNA colonies are then sequenced in situ using the method of WO 98/44152 to identify the Restriction Sequence Tags.

FIG. 12 shows the generation of blunt ends from 3′ overhangs (illustrated for a PstI digest) and partial filling of 5′ overhangs (illustrated for MspI digest) by the Klenow polymerase in presence of dCTP.

### 5. DETAILED DESCRIPTION OF THE INVENTION

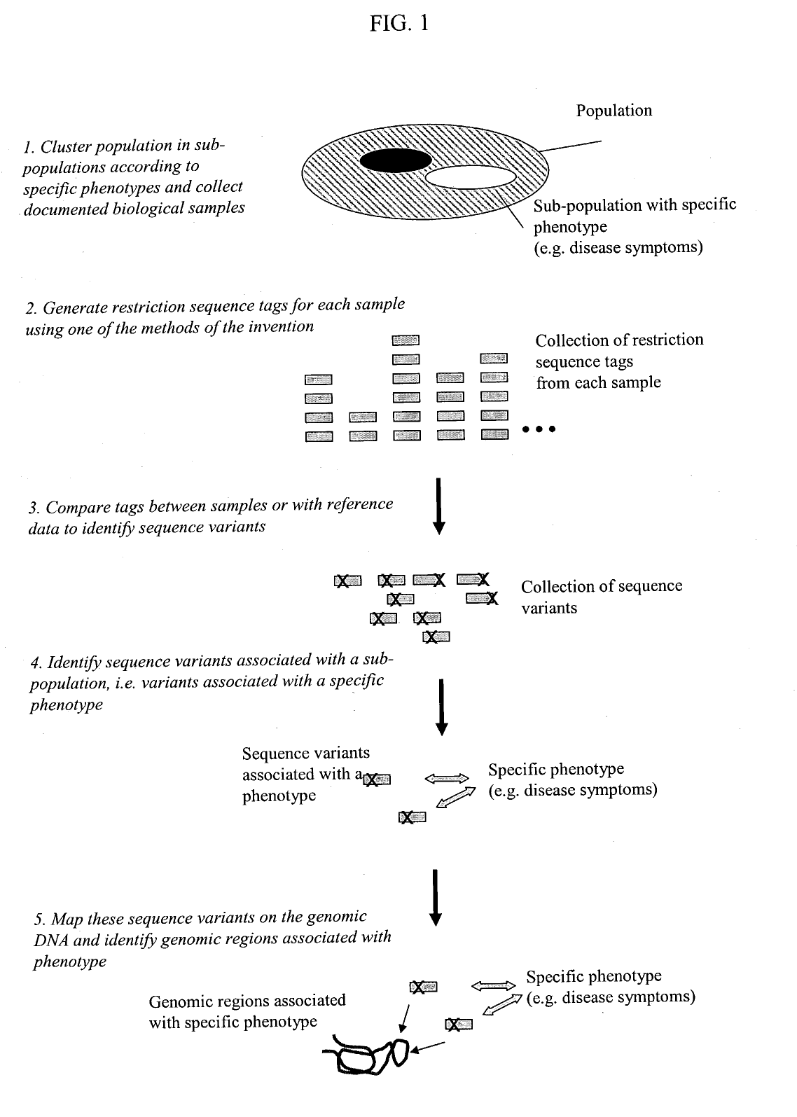

The invention provides methods for determining genome-wide sequence variations associated with a phenotype of a species (see, e.g., FIG. 1). The invention is based at least in part on the discovery that sequence variations associated with a phenotype can be determined hypothesis-free by acquiring and comparing a sufficiently large number of sequence tags from the genomic DNA or cDNAs of individuals who have the phenotype. For example, the genome-wide variations can be determined from a sub-population of individuals of a particular phenotype, e.g. individuals belonging to a particular race, variety, species, genus, family etc., with the same phenotypical characteristics. The genome-wide variations can also be determined from sub-populations of, e.g., healthy individuals, individuals having or susceptible to a particular disease, or individuals at a particular stage of development.

In the methods of the invention, a set of restriction fragments for each member of a sub-population of individuals having the phenotype are generated by digesting nucleic acid from the individual using one or more different restriction enzymes. As used herein, a set of restriction fragments can comprise one or more restriction fragments. A set of restriction sequence tags for the individual is then determined from the set of restriction fragments. The restriction sequence tags for the sub-population of organisms are compared and grouped into one or more groups, each of which comprising restriction sequence tags that comprise homologous sequences. In one embodiment, a group of restriction tags consists of restriction tags that are at least 60%, 70%, 80%, 90%, or 99% homologous. In another embodiment, a group of restriction tags consists of restriction tags that are 100% homologous. The obtained one or more groups of restriction sequence tags can be used to identify the sequence variations associated with the phenotype. In a preferred embodiment, the phenotype under study is associated with proportions or combinations of sequence variations. The invention also provides methods for determining genome-wide sequence variations among a plurality of phenotypes by comparing the restriction sequence tags of different phenotypes. The methods of the invention are applicable to any species of organism. The methods of the invention are particularly useful for higher eukaryotic organisms which have complex genomes, such as higher animals, including but not limited to humans, and plants. In particular, the methods of the invention are useful for analyzing and identifying sequence variations associated with disease susceptibility or response to treatments in a human.

The methods of the present invention can be used to identify polymorphisms in the genome of a species from restriction sequence tags. The methods present several advantages as compared to existing methods: i) it is not necessary to discover a large set of polymorphisms prior to starting a correlation study; ii) it is not necessary to select a limited set of polymorphisms prior to starting a correlation study; iii) it is not necessary to use a priori knowledge of any sequence; iv) it is not necessary to synthesize a large set of different oligonucleotides; v) it is not necessary to perform a large number of specific amplification steps; vi) the number of polymorphisms used in the study can be easily increased by using a large number of different restriction enzymes; vii) the whole procedure is conducted by manipulating a single physical sample whereas in other methods there is at least one step, the amplification step, where the number of physical samples is proportional to the number of polymorphisms to be analyzed; viii) it is not necessary to pool the samples of the population, as each individual can be analyzed; ix) sequence variations existing at very low frequency in the population can be identified; x) the cost of analysis is orders of magnitude cheaper than current genotyping methods.

In the description and examples that follow, a number of terms are used herein. In order to provide a clear and consistent understanding of the specification and claims, including the scope to be given to such terms, the following definitions are provided.

The term “genomic region” refers to a portion of a genome which contains one or a plurality of sequence variations identified by comparing samples from a population of individuals using the methods of the invention.

The term “nucleic acid” refers to at least two nucleotides covalently linked together. A nucleic acid of the present invention can contain phosphodiester bonds. A nucleic acid of the present invention can also be nucleic acid analogs which have a backbone comprising, for example, phosphoramide (see, e.g., Beaucage et al., 1993, Tetrahedron 491925, which is incorporated by reference herein in its entirety), phosphorothioate (see, e.g., Mag et al., 1991, Nucleic Acids Res. 19:1437 and U.S. Pat. No 5,644,048, each of which is incorporated by reference herein in its entirety), phosphorodithioate (see, Briu et al. (1989) J. Am. Chem. Soc. 111:2321), O-methylphophoroamidite linkages (see, e.g., Eckstein, Oligonucleotides and Analogues: A Practical Approach, Oxford University Press), and peptide nucleic acid backbones and linkages (see, e.g., Egholm (1992) J. Am. Chem. Soc. 114:1895; Nielsen (1993) Nature 365:566, all of which are incorporated by reference herein in their entirety). Other analog nucleic acids include those with positive backbones (see, e.g, Denpcy et al (1995) Proc. Natl. Acad. Sci. USA 92:6097,which is incorporated by reference herein in its entirety), non ionic backbones (U.S. Pat. Nos. 5,386,023; 5,637,684; 5,602,240; 5,216,141; and 4,469,863, each of which is incorporated by reference herein in its entirety) and non-ribose backbone including those described in U.S. Pat. Nos. 5,235,033 and 5,034,506, each of which is incorporated by reference herein in its entirety. Nucleic acids containing one or more carbocyclic sugars are also included within the definition of nucleic acids (see, e.g., Jenkins et al. (1995) Chem. Soc. Rev., pp169-176, which is incorporated by reference herein in its entirety). Several nucleic acids analogs are also described in Rawls, C & E News, Jun. 2, 1997, page 3, which is incorporated by reference herein in its entirety. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acids analogs, and mixture of naturally occurring nucleic acids and analogs may be made. A person skilled in the art will know how to select the appropriate analog to use in various embodiments of the present invention. For example, when digesting with restriction enzymes, natural nucleic acids are preferred. The nucleic acids may be single-stranded or double-stranded, as specified, or contain portions of both double-stranded or single-stranded sequence. The nucleic acid may be DNA, e.g., genomic DNA, cDNA, RNA or a hybrid in which the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

The term “oligonucleotide” as used herein includes linear oligomers of natural or modified monomers or linkages, including deoxyribonucleosides, ribonucleosides, and the like, capable of specifically binding to a target polynucleotide by way of a regular pattern of monomer to monomer interactions, such as Watson-Crick type of base pairing, base stacking, Hoogsteen or reverse Hoogsteen types of base pairing, or the like. Preferably, monomers are linked by phosphodiester bonds or analogs thereof to form oligonucleotides ranging in size from a few monomeric units, e.g., 3-4, to several tens of monomeric units, e.g., 40-60. Whenever an oligonucleotide is represented by a sequence of letters, such as “ATGCCTG”, it will be understood that the nucleotides are in 5′ to 3′ order from left to right and that “A” denotes adenosine, “C” denotes citidine, “G” denotes guanosine, “T” denotes thymidine, and “U” denotes uridine, unless otherwise noted. The term “nucleotide” refer to “a deoxyribonucleoside” or “a ribonucleoside,” and “dATP, “dCTP, “dGTP”, “dTTP”, and “dUTP” represent the triphosphate derivatives of the individual nucleotides. Usually oligonucleotides comprise natural nucleotides; however, they may also comprise non-natural nucleotide analogs. It will be clear to those skilled in the art that, although oligonucleotides having natural or non-natural nucleotides may be employed, when, e.g., processing by enzymes is to be carried out, oligonucleotides consisting of natural nucleotides are preferred.

The term “polymorphism” refers to the existence of two or more alleles at in the population. The term “allele” refers to one of several alternative sequence variants at a specific locus. Polymorphism at a single chromosomal location constitutes a genetic marker. The term “SNP” refers to Single Nucleotide Polymorphism. Preferably, a genetic variation, e.g., SNP, is common in a population of organisms and is inherited in a Mendelian fashion. Such alleles may or may not have associated phenotypes.

The term “heterozygote”, as used herein, refers to an individual with different alleles at corresponding loci on homologous chromosomes. Accordingly, the term “heterozygous”, as used herein, describes an individual or strain having different allelic genes at one or more paired loci on homologous chromosomes.

The term “homozygote”, as used herein, refers to an individual with the same allele at corresponding loci on homologous chromosomes. Accordingly, the term “homozygous”, as used herein, describes an individual or a strain having identical allelic genes at one or more paired loci on homologous chromosomes.

The term “mutation” means a heritable alteration in the DNA sequence of an organism.

The term “genotype” is commonly known to mean (i) the genetic constitution of an individual, or (ii) the types of allele found at a locus in an individual.

The term “restriction endonuclease” or “restriction enzyme” refers to an enzyme that recognizes a specific base sequence (a target or recognition site) in a double-stranded DNA molecule and cleaves the DNA molecule at or near, e.g., within a specific distance from, a target or recognition site.

The term “restriction site” refers to a region usually between, but not limited to, 4 and 8 nucleotides, or more than 20 nucleotides, within a nucleic acid, preferably a double-stranded nucleic acid, comprising the recognition site and/or the cleavage site of a restriction endonuclease. A recognition site corresponds to a sequence within a nucleic acid which a restriction endonuclease or group of restriction endonucleases binds to. A cleavage site or cut site corresponds to the particular sequence where cut by the restriction endonuclease occurs. Depending on the restriction endonuclease, the cut site may be within the recognition site. However some restriction endonucleases, e.g., a type-IIS endonuclease, have cleavage sites which are outside the recognition sites.

The term “restriction fragment” refers to a DNA molecule produced by digestion of DNA molecules with a restriction endonuclease.

The term “engineered nucleic acid” or “adaptor” refers to a short double-stranded DNA molecule which has a predetermined nucleotide sequence. Preferably, an engineered nucleic acid or adaptor is 10 to 500 base pairs long. More preferably, an engineered nucleic acid or adaptor is 10 to 150 base pairs long. Preferably, it is designated in such a way that it can be ligated to the ends of restriction fragments. Such nucleic acids can be designed by anyone skilled in the art once the sequence of the ends of restriction fragments is given. Preferably, an engineered nucleic acid comprises sequences of one or more amplification primers, each of which is preferably close to an end of the engineered nucleic acid and oriented to permit primer extension in the direction of towards the end of the molecule. The amplification primers can be the same or different. Preferably, an engineered nucleic acid also comprises sequences of one or more sequencing primers, each of which is preferably close to an end of the engineered nucleic acid and oriented to permit primer extension in the direction of towards the end of the molecule. The sequencing primers can be the same or different. In some embodiments, the amplification primers and sequencing primers can be the same. In some embodiments, an engineered nucleic acid can also comprise one or more restriction sites. An engineered nucleic acid is also referred to as a DNA colony vector in this disclosure.

The term “ligation” refers to an enzymatic reaction catalyzed by a ligase in which two double-stranded DNA molecules are covalently joined together. One or both DNA strands can be covalently joined together. It is also possible to prevent the ligation of one of the two strands through chemical and/or enzymatic modification of one of the ends to permit joining only one of the two DNA strands.

The term “solid support” refers to any solid surface to which nucleic acids can be attached, such as, but not limited to, latex beads, dextran beads, polystyrene, polypropylene surface, polyacrylamide gel, gold surface, glass surfaces and silicon wafers. Preferably, the solid support is a glass surface.

The term “nucleic acid colony” or “colony” refers to a discrete area on, e.g, a solid surface, comprising multiple copies of a nucleic acid strand. Multiple copies of the complementary strand may also be present in the same colony. The multiple copies of the nucleic acid strand making up the colonies are generally immobilized on a solid support and may be in a single or double stranded form.

The term “colony primer” as used herein refers to a nucleic acid molecule which comprises an oligonucleotide sequence which is capable of hybridizing to a complementary sequence and initiate a specific polymerase reaction. The sequence comprising the colony primer is chosen such that it has maximal hybridizing activity with its complementary sequence and very low non-specific hybridizing activity to any other sequence. The colony primer can be 5 to 100 bases in length, but preferably 15 to 25 bases in length. Naturally occurring or non-naturally occurring nucleotides may be present in the primer. One or more than one different colony primers may be used to generate nucleic acid colonies in the methods of the present invention.

5.1. Collecting and Documenting Samples from Individuals of a Particular Phenotype

Genomic DNA or cDNAs of individuals of a particular phenotype can be derived from samples collected from such individuals. Preferably, a sub-population of individuals having the phenotype, e.g. individuals belonging to a particular race, variety, species, genus, family etc., with the same phenotypic characteristics, or individuals having a particular condition, e.g., healthy, having a particular disease, or at a particular stage of development, are identified. Samples from such a sub-population of individuals are collected with detailed documentation of the phenotypic characteristics associated with the sub-population. Such careful documentation facilitates the assignment of sequences variations to one or more phenotypes.

5.2. Method for Generation of Restriction Fragments by Restriction Digestion

The methods of the invention involve generating a set of restriction fragments from genomic DNA or cDNAs from an organism, e.g., genomic DNA extracted from a cell derived from the organism or cDNAs prepared from mRNAs extracted from a cell derived from the organism. In the invention, DNA, e.g., genomic DNA, can be obtained from an individual, e.g. from different cells, parts, tissues or organs. In various embodiments of the invention, one or more different restriction enzymes are employed concurrently or separately to generate the set of restriction fragments from, e.g., genomic DNA. Preferably, the set of restriction fragments comprises a sufficiently large number of different restriction fragments to permit identifying sequence variations in the genome of the organism. More preferably, the set of restriction fragments comprises a least 10, 100, 1000, 10⁴, 10⁵, 10⁶, 10⁷, or 10⁸ different restriction fragments.

The nucleic acid molecules to be analyzed, e.g., genomic DNA, can be obtained from any source, e.g., tissue homogenate, blood, amniotic fluid, chorionic villus samples, and bacterial culture. The nucleic acid molecules can be obtained from these sources using standard methods known in the art. Preferably, only a minute quantity of nucleic acid is required, which can be DNA or RNA (in the case of RNA, a reverse transcription step is required before the PCR step). The molecular biology methods, if used in a method of the present invention, are carried out using standard methods (e.g., Ausubel et al., Current Protocols in Molecular Biology, John Wiley and Sons, New York 1989; Sambrook et al., Molecular Cloning, Laboratory Manual, 3^{rd} Editions, Cold Spring Harbor N.Y., 2001; Innis et al., PCR Protocols: A Guide to Methods and Applications, Academic Press, Cold Spring Harbor N.Y., 1989).

Any restriction enzymes known in the art can be used in conjunction with the present invention. In some embodiments of the invention Type-IIS endonucleases are used in one or more steps. Type-IIS endonucleases are generally commercially available and are well known in the art. A Type-IIS endonuclease recognizes a specific sequence of base pairs within a double stranded polynucleotide sequence. Upon recognizing that sequence, the endonuclease will cleave the polynucleotide sequence, generally leaving an overhang of one strand of the sequence, or “sticky end.” Type-IIS endonucleases do not require that the specific recognition site be palindromic like those of the type-II endonucleases, i.e., when reading in the 5′ to 3′ direction, the base pair sequence being the same for both strands of the recognition site. Additionally, Type-IIS endonucleases also generally cleave outside of their recognition sites. Because the cleavage occurs in a location of any polynucleotide sequence a certain base pairs away from the recognition site, a Type-IIS permits the capturing of the intervene sequence up to the cleavage site in some embodiments of the present invention. Specific Type-IIs endonucleases which are useful in the present invention include, but are not limited to, EarI, MnlI, PleI, AlwI, BbsI, BceAI, BsaI, BsmAI, BspMI, Eco57I, Esp3I, HgaI, SapI, SfaNI, BbvI, BsmFI, FokI, BseRI, HphI, MmeI and MboIL. Currently discovered enzymes cut a maximum of 20-25 bases from their recognition site. Enzymes cutting further away, for instance at more than 50, 100 or more than 200 bases from their recognition site would be useful for the invention.

In some embodiments of the invention, rare cutter and frequent cutter combinations are used to generate the restriction fragments. A rare cutter is a restriction endonuclease which has a recognition site consisting of a sequence of more than four nucleotides, preferably 6 or 8 nucleotides. Examples of commercially available rare cutters are PstI, HpaII, MspI, ClaI, HhaI, EcoRII, BstBI, HinP1, MaeII, BbvI, PvuII, XmaI, SmaI, NciI, AvaI, HaeII, SalI, XhoI and PvuII, of which PstI, HpaII, MspI, ClaI, HhaI, EcoRII, BstBI, HinP1, and MaeII are preferred. A frequent cutter is a restriction endonuclease which has a four-base or less-than-four-base nucleotide recognition site. Examples of suitable frequent cutter enzymes include MseI and TaqI.

In some embodiments of the invention, restriction fragments are linked to other nucleic acids or to themselves at the digestion sites. Typically, restriction enzymes produce either blunt ends, in which the terminal nucleotides of both strands are base paired, or staggered ends, in which one of the two strands protrudes to give a short single stranded extension. In some embodiments of the invention, when the restriction enzyme is a Type-IIS, a step which comprises the modification of the ends by converting protruding ends into blunt ends with a polymerase is preferably added.

5.3. Methods for Determination of Restriction Sequence Tags

Any method known in the art can be used to determine a set of restriction sequence tags for the restriction fragments generated by a method of Section 5.2. Preferably, the restriction fragments are amplified before sequencing. However, sequencing methods that do not require amplification, such as single-molecule sequencing, can also be used without an additional amplification step. Preferably, the lengths of the restriction sequence tags generated are at least 5 nucleotides. More preferably, the restriction sequence tags generated are at in the range of 10 to 20 nucleotides. Still more preferably, the lengths of the restriction sequence tags are up to 50 nucleotides.

Preferably, a method which involves generation and sequencing of DNA colonies is used to determine the restriction sequence tags of the restriction fragments. Any one of the methods known in the art can be used in the present invention (see, e.g., PCT publications WO 98/44151, WO 98/44152, WO 00/18957, and WO 02/46456, all of which are incorporated by reference herein in their entirety). One nucleic acid colony can be generated from a single immobilized nucleic acid template, e.g., a nucleic acid template derived from a restriction fragment. The methods of the invention allow the simultaneous production of a number of such nucleic acid colonies, each of which contain a different immobilised nucleic acid.

DNA colonies can be generated by a method comprising capturing and amplifying DNA fragments, e.g., restriction fragments, using primers immobilized on a solid surface (see, PCT publications WO 98/44151 and WO 98/44152). In embodiments of the invention in which DNA fragments are circular, a step of linearizing the circular DNA fragments using a restriction enzyme is preferably performed before colony generation. In one embodiment, DNA colonies are generated from a sample of DNA molecules, e.g, a pool of restriction fragments, by a method comprising the steps of:

i) providing a solid surface comprising a plurality of colony primers immobilized on said solid surface at 5′ end, wherein each colony primer comprises a sequence that is hybridizable to a sequence at the 3′ end of the DNA molecules in the sample;

ii) denaturing the DNA molecules to generate single stranded fragments;

iii) annealing the single stranded fragments to the immobilized colony primers;

iv) carrying out primer extension reaction using the annealed single stranded fragments as templates to generate immobilized double stranded nucleic acid fragments;

v) denaturing the immobilized double stranded nucleic acid fragments to generate immobilized single stranded fragments;

vi) annealing the immobilized single stranded fragments to immobilized colony primers;

vii) repeating the steps iv) through vi) such that the colonies are generated, each at a particular location on the solid surface.

In a preferred embodiment, the immobilized colony primers comprise a sequence that is hybridizable to a sequence in the DNA molecules. For example, the DNA molecules in the sample can be restriction fragments linked to a nucleic acid having a predetermined sequence. In such a case, immobilized primers can have a sequence that is hybridizable to a sequence in the predetermined sequence. In some other embodiments of the invention, colony primers having different sequences can be used. Primers for use in the present invention are preferably at least five bases long. More preferably, the primers are less than 100 or less than 50 bases long. The present invention uses repeated steps of annealing of templates to immobilized primers, primer extension and separation of extended primers from templates. It will be appreciated by those skilled in the art that these steps can be performed using reagents and conditions in PCR (or reverse transcriptase plus PCR) techniques. PCR techniques are disclosed, for example, in “PCR: Clinical Diagnostics and Research”, published in 1992 by Springer-Verlag, which is incorporated herein by reference in its entirety.

DNA colonies can also be generated by a method as described in PCT Publication WO 00/18957. In embodiments of the invention in which DNA fragments to be amplified are circular, a step of linearizing the circular DNA fragments using a restriction enzyme is preferably performed before colony generation. In one embodiment, DNA colonies are generated from a sample of DNA molecules, e.g, a pool of restriction fragments, by a method comprising the steps of:

i) mixing the DNA molecules in the sample with colony primers, wherein each colony primer comprises a sequence that is hybridizable to a sequence at the 3′ end of the DNA molecules;

ii) grafting the DNA molecules and colony primers on a solid surface at the 5′ ends of both the DNA molecules and colony primers to generate immobilized DNA molecules and immobilized colony primers;

iii) denaturing said immobilized DNA molecules to generate immobilized single-stranded fragments;

iv) annealing said immobilized single stranded fragments to immobilized colony primers to obtain annealed single-stranded fragments;

v) carrying out primer extension reactions using said annealed single stranded fragments as templates to generate immobilized double stranded nucleic acid fragments;

vi) denaturing the immobilized double stranded nucleic acid fragments to generate immobilized single stranded fragments;

vii) annealing the immobilized single stranded fragments to immobilized colony primers; and

viii) repeating the steps iv) through vii) such that the colonies are generated, each at a particular location on the solid surface.

Preferably the proportion of colony primers in the mixture is higher than the proportion of colony templates. Preferably the ratio of colony primers to colony templates is such that when the colony primers and nucleic acid templates are immobilised to the solid support a “lawn” of colony primers is formed comprising a plurality of colony primers being located at an approximately uniform density over the whole or a defined area of the solid support, with one or more colony templates being immobilized individually at intervals within the lawn of colony primers. Primers for use in the present invention are preferably at least five bases long. More preferably, the primers are less than 100 or less than 50 bases long. The present invention uses repeated steps of annealing of templates to immobilized primers, primer extension and separation of extended primers from templates. It will be appreciated by those skilled in the art that these steps can be performed using reagents and conditions in PCR (or reverse transcriptase plus PCR) techniques. PCR techniques are disclosed, for example, in “PCR: Clinical Diagnostics and Research”, published in 1992 by Springer-Verlag.

Isothermal amplification of nucleic acids on a solid support can also be used to generated DNA colonies (see, e.g., PCT publication WO 02/46456). In embodiments of the invention in which DNA fragments to be amplified are circular, a step of linearizing the circular DNA fragments using a restriction enzyme is preferably performed before colony generation. In one embodiment, DNA colonies are generated from a sample of DNA molecules, e.g, a pool of restriction fragments, by a method comprising the steps of:

i) mixing DNA molecules in the sample with colony primers, wherein each colony primer comprises a sequence that is hybridizable to a sequence at the 3′ end of the DNA molecules, and wherein the concentration of the colony primers is adjusted such that amplification of grafted DNA molecules can occur;

ii) grafting the DNA molecules and colony primers on a solid surface at the 5′ end to generate immobilized DNA molecules and immobilized colony primers;

iii) applying an amplification solution containing a polymerase and nucleotides to the solid surface such that the colonies are generated isothermally, each at a particularly location on the solid surface.

The quantity of immobilized nucleic acids in step ii) determines the average number of DNA colonies per surface unit which can be created. The ranges of preferred concentrations of the DNA molecules to be immobilized are preferably between 1 nanoMolar and 0.01 nanoMolar for the colony templates, and between 50 and 1000 nanoMolar for the colony primers. In a preferred embodiment, the temperature of the reaction is chosen to be the optimal temperature for the polymerase activity. In preferred embodiments, the DNA molecules in the sample have sizes in the range of about 50-5000 base pairs.

In the methods described in this section, colonies are generated on discrete locations on the surface. Densities of colonies on a surface can be controlled by, e.g., adjusting the density of primers immobilized on the surface. In preferred embodiments, colony densities are 10⁴⁻⁶ colonies/cm², more preferably 10⁷⁻⁸ colonies/cm² or more. The size of colonies can also be controlled by adjusting the experimental conditions. Preferably colonies measure from 10 nm to 100 μm across their longest dimension, more preferably from 100 nm to 10 μm across their longest dimension.

DNA colonies can be sequenced to determine at least a portion of their sequences. In one embodiment, sequencing is carried out by hybridizing an appropriate primer, sometimes referred to herein as a “sequencing primer”, with the nucleic acid molecules in DNA colonies, extending the primer and detecting the nucleotides used to extend the primer. Preferably the nucleotide used to extend the primer in each colony is detected before the next nucleotide is added to the growing nucleic acid chain, thus allowing base by base in situ nucleic acid sequencing.

The detection of incorporated nucleotides is facilitated by including one or more labeled nucleotides in the primer extension reactions. Any appropriate detectable label may be used, for example a fluorophore, a radioactive label etc. Preferably a fluorescent label is used. Any fluorescent label known in the art can be used. The same or different labels may be used for each different type of nucleotide. Where the label is a fluorophore and the same labels are used for each different type of nucleotide, each nucleotide incorporation provides a cumulative increase in signal detected at a particular wavelength. If different labels are used, these signals may be detected at different appropriate wavelengths. In a preferred embodiment, a mixture of labelled and unlabelled nucleotides of the same type are used for each primer extension step.

In order to allow the hybridization of an appropriate sequencing primer to the nucleic acid template to be sequenced the nucleic acid template should normally be in a single stranded form. If the nucleic acid templates making up the nucleic acid colonies are present in a double stranded form, they can be processed to provide single stranded nucleic acid templates using methods well known in the art, for example, but not limited to, by denaturation, cleavage etc.

The sequencing primers which are hybridized to the nucleic acid template and used for primer extension are preferably short oligonucleotides, for example of 15 to 25 nucleotides in length. The sequence of the primers can be designed so that they hybridize to part of the nucleic acid template to be sequenced, preferably under stringent conditions. The sequence of the primers used for sequencing may have the same or similar sequences to that of the colony primers used to generate the nucleic acid colonies.

Once the sequencing primer has been annealed to the nucleic acid template to be sequenced by subjecting the nucleic acid template and sequencing primer to appropriate conditions, determined by methods well known in the art, primer extension is carried out, for example using a nucleic acid polymerase and a supply of nucleotides, at least some of which are provided in a labelled form, and conditions suitable for primer extension if a suitable nucleotide is provided. DNA polymerases and nucleotides which may be used are well known to one skilled in the art.

Preferably after each primer extension step a washing step is included in order to remove unincorporated nucleotides which may interfere with subsequent steps. After a primer extension step has been carried out the DNA colony can be detected in order to determine whether a labelled nucleotide has been incorporated into an extended primer. The primer extension step may then be repeated in order to determine the next and subsequent nucleotides incorporated into an extended primer.

Any device allowing detection the presence or absence, and preferably the amount, of the appropriate label incorporated into an extended primer, for example fluorescence or radioactivity, may be used for sequence determination. In an embodiment in which the label is a fluorescence label, a CCD camera attached to a magnifying device (such as a microscope), may be used.

The detection system is preferably used in combination with an analysis system in order to determine the number and identity of the nucleotides incorporated at each colony after each step of primer extension. This analysis, which may be carried out immediately after each primer extension step, or later using recorded data, allows the sequence of the nucleic acid template within a given colony to be determined.

In a further embodiment of the present invention, the full or partial sequence of more than one nucleic acid can be determined by determining the full or partial sequence of the nucleic acid templates present in more than one nucleic acid colony. Preferably a plurality of sequences are determined simultaneously and the nucleotides applied to nucleic acid colonies are usually applied in a chosen order which is then repeated throughout the analysis, for example dATP, dTTP, dCTP, dGTP.

Thus it can be seen that full or partial sequences of the nucleic acid templates making up particular nucleic acid colonies may be determined.

The primers and oligonucleotides used in the methods of the present invention are preferably DNA, and can be synthesized using standard techniques and, when appropriate, detectably labeled using standard methods (Ausubel et al., supra). Detectable labels that can be used in the method s of the present invention include, but are not limited to, fluorescent labels (e.g. fluorescein and rhodamin). The labels used in the methods of the invention are detected using standard methods.

The methods of the invention can also be facilitated by the use of kits which contain reagents required for carrying out the assays. The kits can contain reagents for carrying out the analysis of a single restriction fragment tag (for use in, e.g., diagnostic methods) or multiple restriction fragment tags (for use in, e.g., genomic mapping). When multiple samples are analyzed, multiple sets of the appropriate primers and oligonucleotides are provided in the kit. In addition, to the primers and oligonucleotides required for carrying out the various methods, the kit may contain the enzymes used in the methods, and the reagents for detecting the labels, etc. The kits can also contain solid substrates for used in carrying out the method of the invention. For example, the kits can contain solid substrates, such as glass plates or silicon or glass microchips.

5.4. Methods for Identifying Restriction Sequence Tags Associated with a Phenotype

The restriction sequence tags obtained for each individual are then compared among the sub-population of a given phenotype to identify all the homologous tags and determine the number of homologous restriction sequence tag. In a preferred embodiment, the two restriction sequence tags obtained within a DNA colony represent the ends of the corresponding restriction fragment in the set of restriction fragments. The two tags originated from locations physically close to each other on the genome. Each tag can also be combined with the sequence of the restriction site of the restriction enzyme used for digestion of the genomic DNA to obtain a longer sequence. Homologous tags are grouped. In one embodiment, a group of restriction tags consists of restriction tags that are at least 60%, 70%, 80%, 90%, or 99% homologous. In another embodiment, a group of restriction tags consists of restriction tags that are 100% homologous. The collection of the groups of restriction tags for a sub-population can be used to identify sequence variations associated with the phenotype. In a preferred embodiment, the phenotype under study is associated with proportions of sequence variations in a population or with combinations of sequence variations. In one embodiment, the proportions of one or more particular sequences in the population, e.g., as represented by the relative numbers of restriction tags in the respective one or more particular groups of restriction sequence tags, each of which is different by more than 10%, 20%, 50%, 70% or 90% between two different populations, are identified as being associated with the phenotypic difference between the two populations. In another preferred embodiment, the phenotype is associated with particular combinations of sequence variations found in individuals from the population. In one embodiment, the combination of proportions of a plurality of particular sequences in the population, e.g., as represented by a combination of the numbers of restriction tags in a plurality of particular groups of restriction sequence tags, i.e., the total number of restrictions tags in the plurality of groups, are identified as being associated with the phenotypic difference between the two populations, if such combination of proportions are different by more than 10%, 20%, 50%, 70% or 90% between the two different populations. In another embodiment, a plurality of such combinations are used to identify the phenotypic difference. In the embodiment where a plurality of combinations is used, each combination in the plurality of combinations can include one or more particular sequences which also included in a different combination in the plurality of the combinations. These embodiments are illustrated in Example 6.3., infra.

In one embodiment, the restriction sequence tags can be compared with the genomic sequence of the organism to identify the genomic locations of the restriction sequence tags. In another embodiment, the restriction sequence tags flanking the genome on both sides of the recognition site are identified from the genomic sequence of the organism.

5.5. Specific Preferred Embodiments for Obtaining Restriction Sequence Tags

Several preferred embodiments for obtaining restriction sequence tags are described in this section. These methods can be used in conjunction with any methods described in Sections 5.1 through 5.4 for identifying sequence variations associated with a phenotype. It will be apparent to one skilled in the art that any repetition and/or combination of one or more of the specific embodiments described in this section can also be used.

(I) First Specific Embodiment

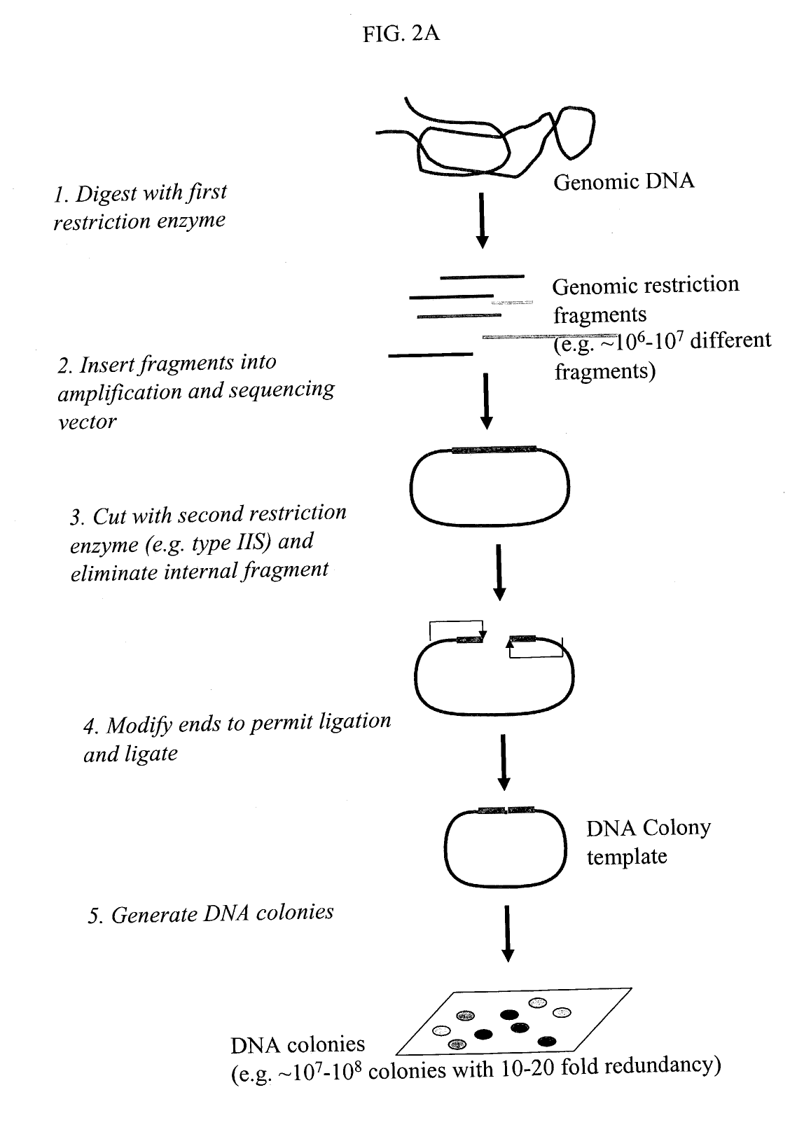

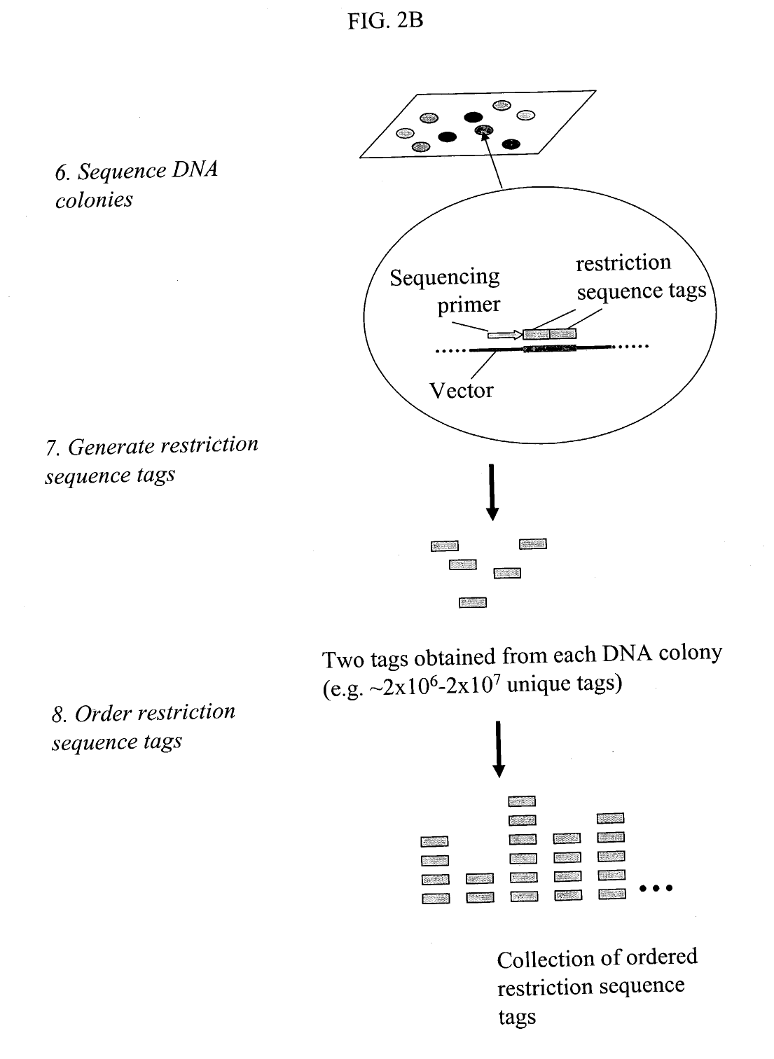

In a preferred embodiment, the invention provides a method for generating restriction sequence tags of a biological sample (FIGS. 2A and 2B). In the method, one or more first restriction enzymes are used to digest the nucleic acids extracted from the biological sample to generate a set of restriction fragments. A set of restriction sequence tags is then determined from the set of restriction fragments by a method comprising the step of:

1) linking restriction fragments in the set of restriction fragments with a first engineered nucleic acid which comprises a predetermined sequence comprising one or more recognition sites of a second restriction enzyme to obtain a set of first circular nucleic acid fragments, the recognition sites being located and oriented such that the second restriction enzyme cuts in the restriction fragments;

2) digesting the first circular nucleic acid fragments with the second restriction enzyme;

3) modifying the ends generated by the second restriction enzyme to permit ligation;

4) linking the ends generated by the second restriction enzyme to produce a set of second circular nucleic acid fragments; and

5) sequencing at least a portion of each of said restriction fragments in the second circular nucleic acids to determine a set of restriction sequence tags.

Preferably, each of the recognition sites of the second restriction enzyme in the first engineered nucleic acid is located close to an end of the first engineered nucleic acid. In one preferred embodiment, each of the recognition site of the second restriction enzyme in the first engineered nucleic acid is located less than 20 nucleotides from an end of the first engineered nucleic acid. More preferably, each of the recognition site of the second restriction enzyme in the first engineered nucleic acid is located zero to 5 nucleotides from an end of the first engineered nucleic acid. Preferably, the second restriction enzyme is a type IIs endonuclease. In a preferred embodiment, the type IIs endonuclease cuts more than 5, 10, 20, 50, 100, or more than 200 bases from its recognition site. In another embodiment, the second circular nucleic acid fragments can be linerized by, e.g., using a third restriction enzyme which is different from the first and the second restriction enzyme, to obtain a set of third restriction fragments. In a preferred embodiment, the method further comprises a step of amplifying the third restriction fragments using primers found in the first engineered nucleic acid. In another preferred embodiment, the step of digesting with a third restriction enzyme and subsequent amplification can be replaced by a step of amplification of the second circular nucleic fragments.

In preferred embodiments, a step of fixing and amplifying the second circular nucleic acid fragments is carried out before step 5). In more preferred embodiments, the fixing and amplifying is carried out by any one of the DNA colony methods described in Section 5.3. In still more preferred embodiments, the sequencing is carried out by one of the base by base primer extension methods described Section 5.3.

In still other preferred embodiments of the invention, the step of modifying said ends of said second restriction fragments is done by filling-in the ends or removing the overhanging nucleotides of said second restriction fragments with a DNA polymerase such that the ends are blunt in order to be linked.

In another preferred embodiment, the method of the invention comprises a purification step and/or DNA isolation step after each step.

In still another preferred embodiment, the small genomic DNA sequences in the set of restriction fragments are linked together up to a certain extent, inserted into a plasmid, cloned into a bacteria, the bacteria plated on an agarose plate and the plasmid of each individual bacteria colony isolated, and sequenced using Sanger sequencing with an automated capillary sequencer. Other approaches that do not use the bacterial cloning step are also known to those skilled in the art. For instance, the first engineered nucleic acid may comprise a combinatorial sequence tag such that the third nucleic acid fragments can be used for molecular cloning on beads and sequenced base by base.

(II) Second Specific Embodiment

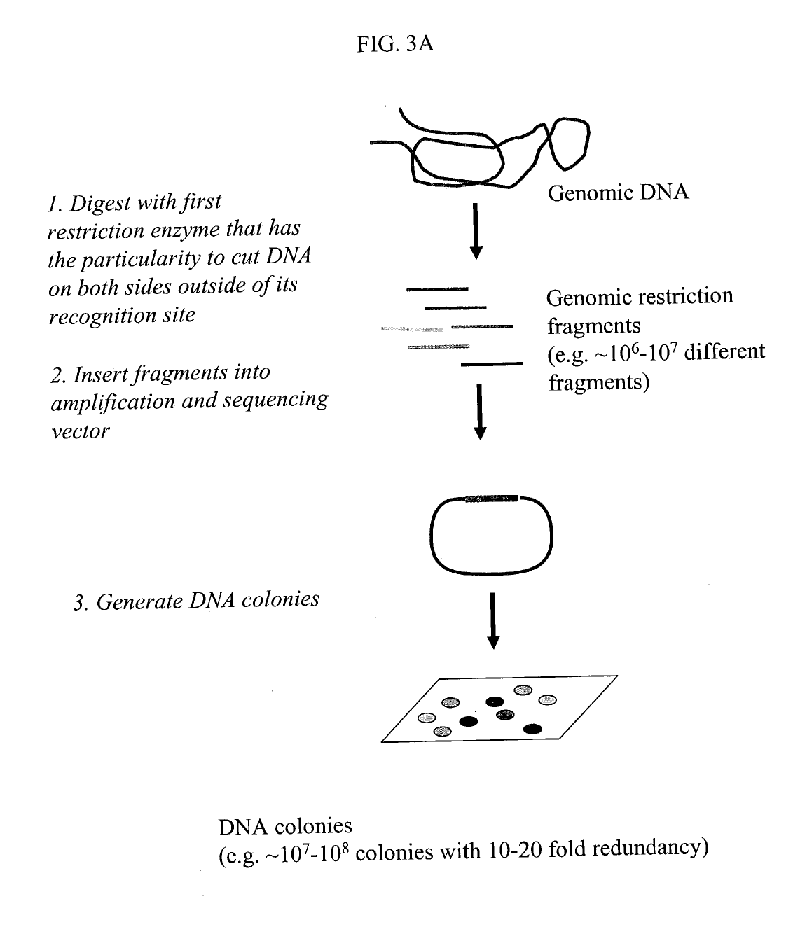

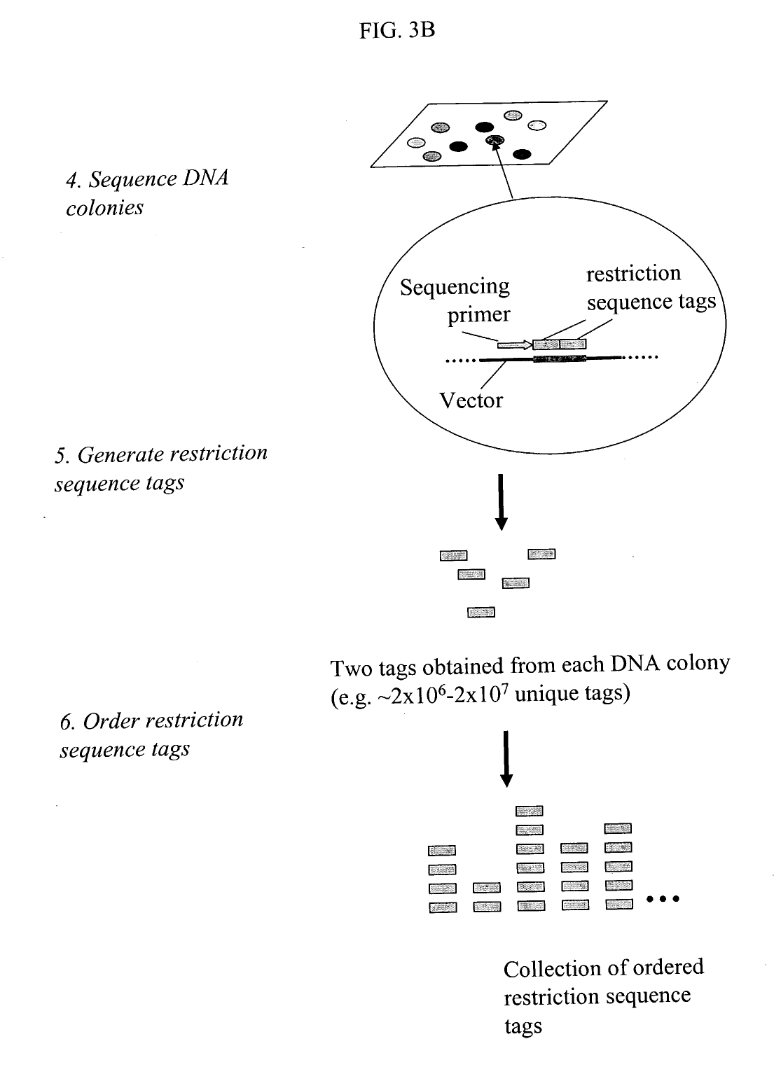

In another embodiment, the invention provides a method for generating restriction sequence tags of a biological sample (FIGS. 3A and 3B). In the method, a first restriction enzyme is used to digest the nucleic acids extracted from the biological sample to generate a set of restriction fragments. The first restriction enzyme cuts at both sides of its recognition site in such a manner that the cutting sites enclose a part of sequence that is not part of the recognition site. Restriction enzymes can be used for this purpose include, but not limited to, BaeI, BcgI, BsaXI. A set of restriction sequence tags is then determined from the set of restriction fragments by a method comprising the step of:

1) modifying the ends generated by the first restriction enzyme to permit ligation;

2) linking the restriction fragments in the set of restriction fragments with a first engineered nucleic acid to obtain a set of first circular nucleic acid fragments, the first engineered nucleic acid comprising a predetermined nucleotide sequence; and

3) sequencing at least a portion of each of the restriction fragments in the first circular nucleic acids to determine the set of restriction sequence tags.

In preferred embodiments, a step of fixing and amplifying the first circular nucleic acid fragments is carried out before step 3). In more preferred embodiments, the fixing and amplifying is carried out by any one of the DNA colony methods described Section 5.3. In still more preferred embodiments, the sequencing is carried out by a base by base primer extension method described Section 5.3.

In still other preferred embodiments of the invention, the step of modifying said ends of said second restriction fragments are done by fill-in the ends or removing the overhanging nucleotides of said second restriction fragments with a DNA polymerase such that the ends are blunt in order to be linked.

In another preferred embodiment, the method of the invention comprises purification step and/or DNA isolation steps after each step.

(III) Third Specific Embodiment

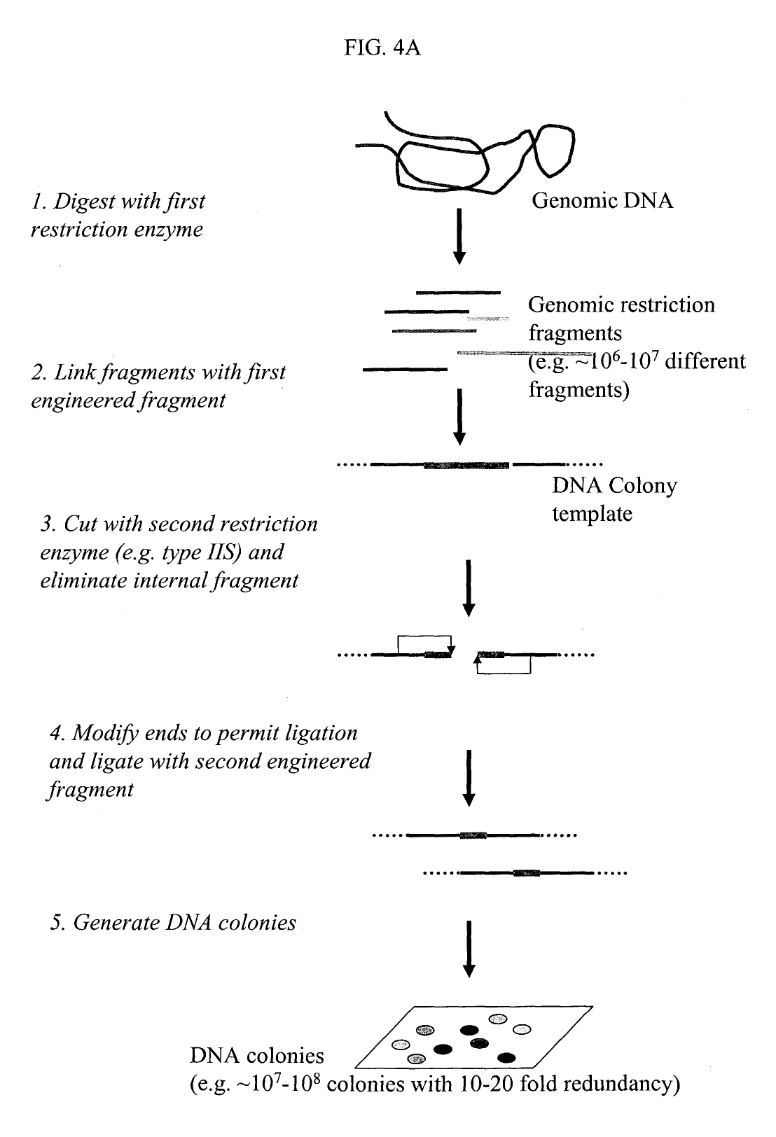

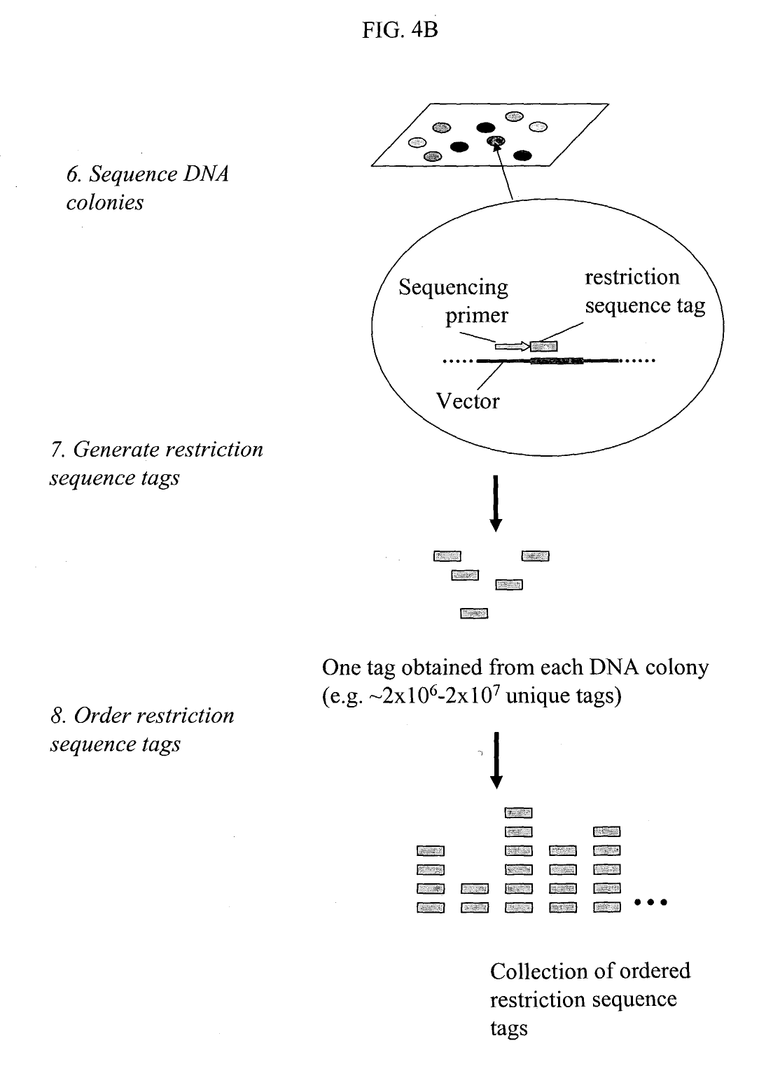

In still another embodiment, the invention provides a method for generating restriction sequence tags of a biological sample (FIGS. 4A and 4B). In the method, one or more first restriction enzymes are used to digest the nucleic acids extracted from the biological sample to generate a set of restriction fragments. A set of restriction sequence tags is then determined from the set of restriction fragments by a method comprising the step of:

1) linking said restriction fragments in the set of restriction fragments with a first engineered nucleic acid to obtain a set of first nucleic acid fragments, the first engineered nucleic acid comprising a predetermined nucleotide sequence comprising a recognition site of a second restriction enzyme, the recognition site being located and oriented such that the second restriction enzyme cuts in the restriction fragments;

2) digesting the first nucleic acid fragments with the second restriction enzyme;

3) modifying the ends generated by the second restriction enzyme to permit ligation

4) linking the ends generated by the second restriction enzyme with a second engineered nucleic acid to produce second nucleic acid fragments, the second engineered nucleic acid comprising a predetermined nucleotide sequence; and

5) sequencing at least a portion of each of the restriction fragments in the second nucleic acid fragments to determine the set of restriction sequence tags.

Preferably, the recognition site of the second restriction enzyme in the first engineered nucleic acid is located close to an end of the first engineered nucleic acid. In one preferred embodiment, the recognition site of the second restriction enzyme in the first engineered nucleic acid is located less 20 nucleotides from an end of the first engineered nucleic acid. In a more preferred embodiment, the recognition site of the second restriction enzyme in the first engineered nucleic acid is located zero to 5 nucleotides from an end of the first engineered nucleic acid. Preferably, the second restriction enzyme is a type IIs endonuclease. In a preferred embodiment, the type IIs endonuclease cuts more than 5, 10, 20, 50, 100, or more than 200 bases from its recognition site.

In preferred embodiments, a step of fixing and amplifying the second nucleic acid fragments is carried out before step 5). In more preferred embodiments, the fixing and amplifying is carried out by any one of the DNA colony methods described Section 5.3. In still more preferred embodiments, the sequencing is carried out by a base by base primer extension method described Section 5.3.

In still other preferred embodiments of the invention, the step of modifying said ends of said second restriction fragments are done by fill-in the ends or removing the overhanging nucleotides of said second restriction fragments with a DNA polymerase such that the ends are blunt in order to be linked.

In another preferred embodiment, the method of the invention comprises purification step and/or DNA isolation steps after each step.

(IV) Fourth Specific Embodiment

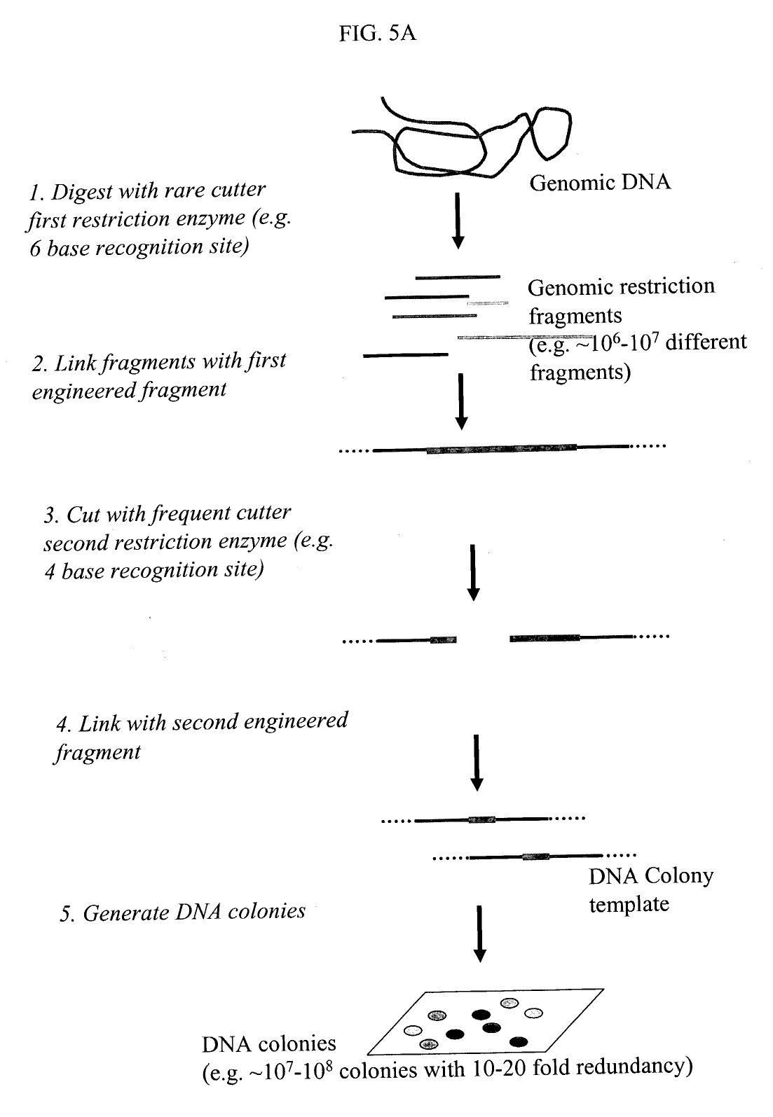

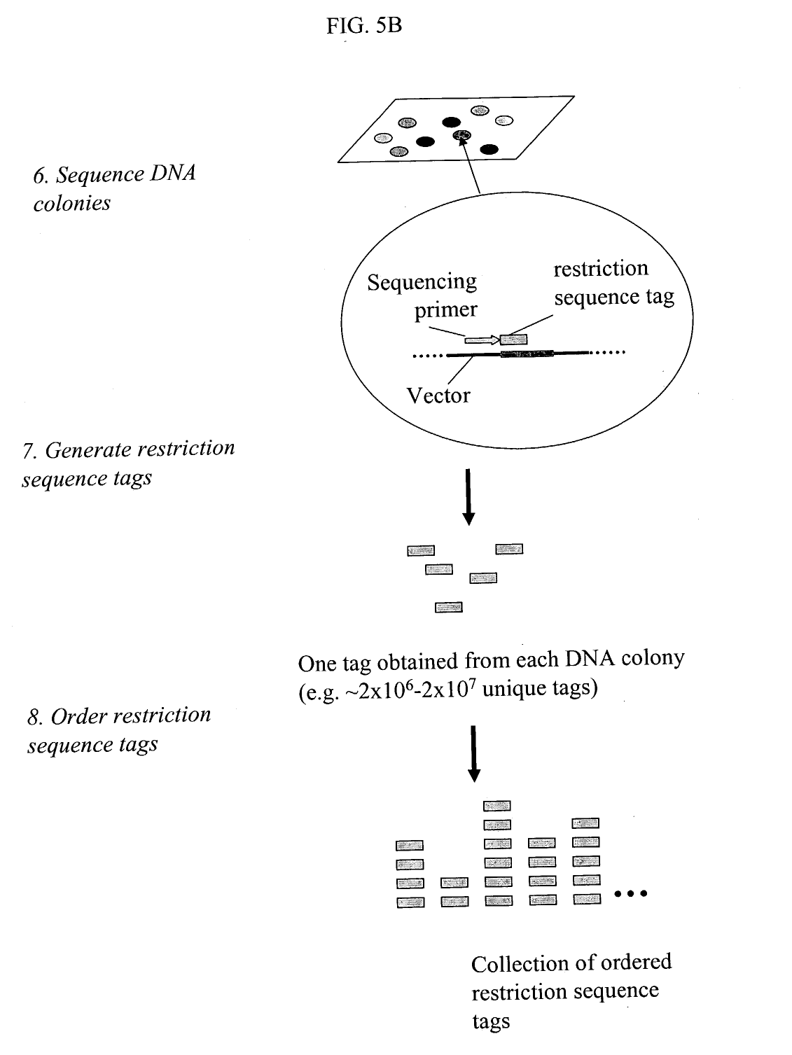

In still another preferred embodiment, the invention provides a method for generating restriction sequence tags of a biological sample (FIGS. 5A and 5B). In the method, one or more rare cutters are used to digest the nucleic acids extracted from the biological sample to generate a set of restriction fragments. Preferably, a rare cutter that recognizes a 6-base, 8-base, or more than-8-base recognition sequence is used. A set of restriction sequence tags is then determined from the set of restriction fragments by a method comprising the step of:

1) linking the restriction fragments in the set of restriction fragments with a first engineered nucleic acid to obtain a set of first nucleic acid fragments, the first engineered nucleic acid comprising a predetermined nucleotide sequence;

2) digesting the first nucleic acid fragments with one or more second restriction enzymes to obtain second restriction fragments, wherein the second restriction enzymes are different from the first restriction enzyme and do not cut in the first engineered nucleic acid;

3) linking the ends of the second restriction fragments with a second engineered nucleic acid to produce a set of second nucleic acid fragments, the second engineered nucleic acid comprising a predetermined nucleotide sequence; and

4) sequencing at least a portion of each of the restriction fragments in the second nucleic acid fragments to determine the set of restriction sequence tags.

In a preferred embodiment, the digestion with the first and second restriction enzymes is performed simultaneously before ligation with first and second engineered fragments.

In preferred embodiments, a step of fixing and amplifying the second nucleic acid fragments is carried out before step 4). In more preferred embodiments, the fixing and amplifying is carried out by any one of the DNA colony methods described Section 5.3. In still more preferred embodiments, the sequencing is carried out by a base by base primer extension method described Section 5.3.

In another preferred embodiment, the method of the invention comprises purification step and/or DNA isolation steps after each step.

(V) Other Specific Embodiments

The invention also provides methods for generating restriction sequence tags of a biological sample. In such methods, one or more first restriction enzymes are used to digest the nucleic acids extracted from the biological sample to generate a set of restriction fragments. A plurality of different second restriction enzymes are then used to further digest the restriction fragments. Such methods permit further increasing the number of restriction sequence tags located close to the recognition sites of the first restriction enzymes.

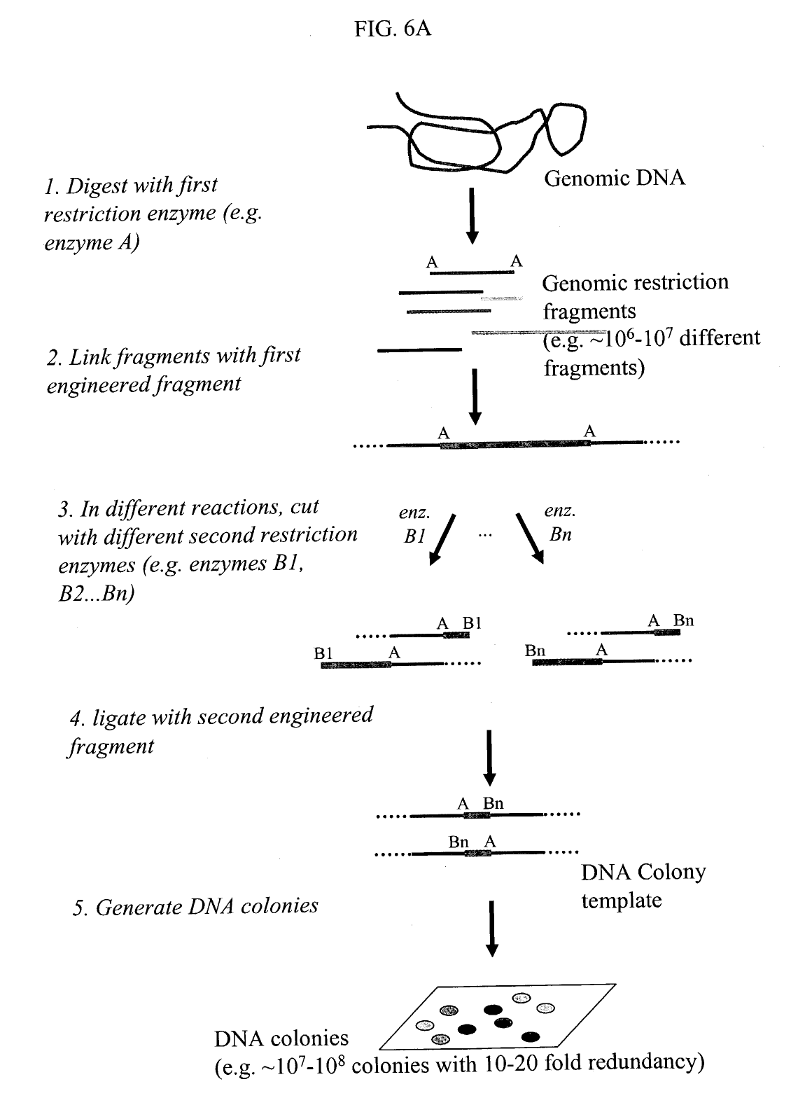

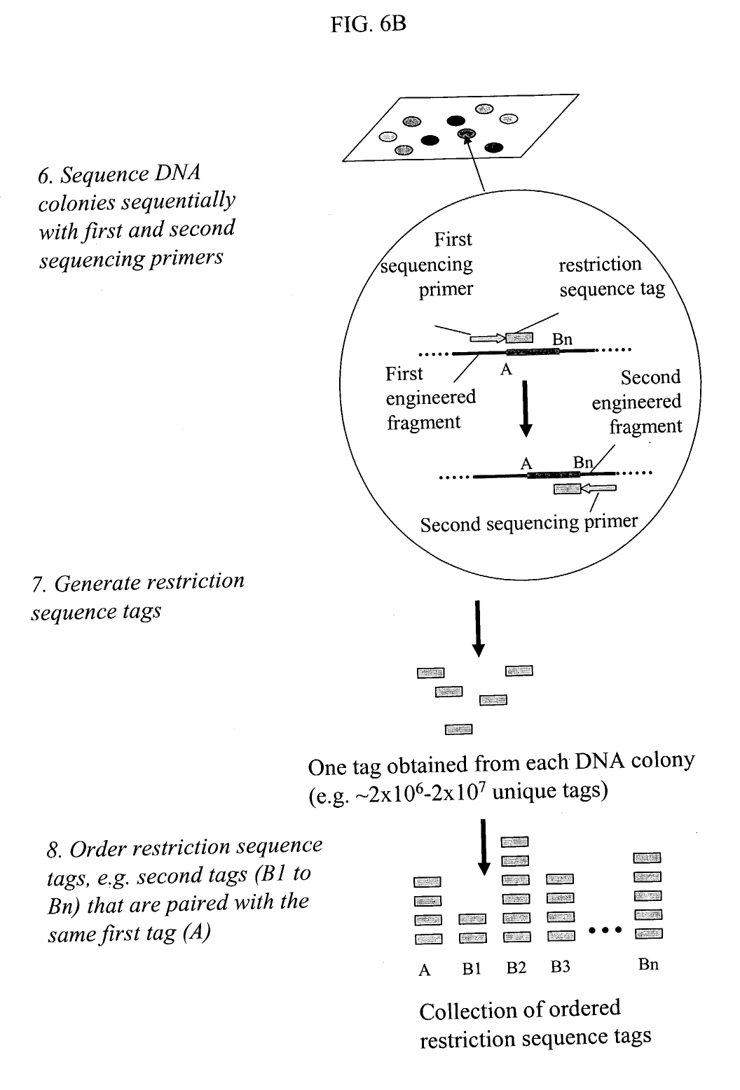

In one preferred embodiment (FIGS. 6A and 6B), after digestion by a first restriction enzyme, a set of restriction sequence tags is determined from the set of restriction fragments by a method comprising the step of:

1) linking said restriction fragments in the set of restriction fragments with a first engineered nucleic acid to obtain a set of first nucleic acid fragments, the first engineered nucleic acid comprising a predetermined nucleotide sequence;

2) digesting the first nucleic acid fragments with a second restriction enzyme to obtain second restriction fragments, wherein the second restriction enzyme is different from the first restriction enzyme and does not cut in the first engineered nucleic acid;

3) linking the ends of the second restriction fragments with a second engineered nucleic acid to produce a set of second nucleic acid fragments, the second engineered nucleic acid comprising a predetermined nucleotide sequence; and

4) sequencing at least a portion of each of the restriction fragments in the second nucleic acid fragments to determine the set of restriction sequence tags.

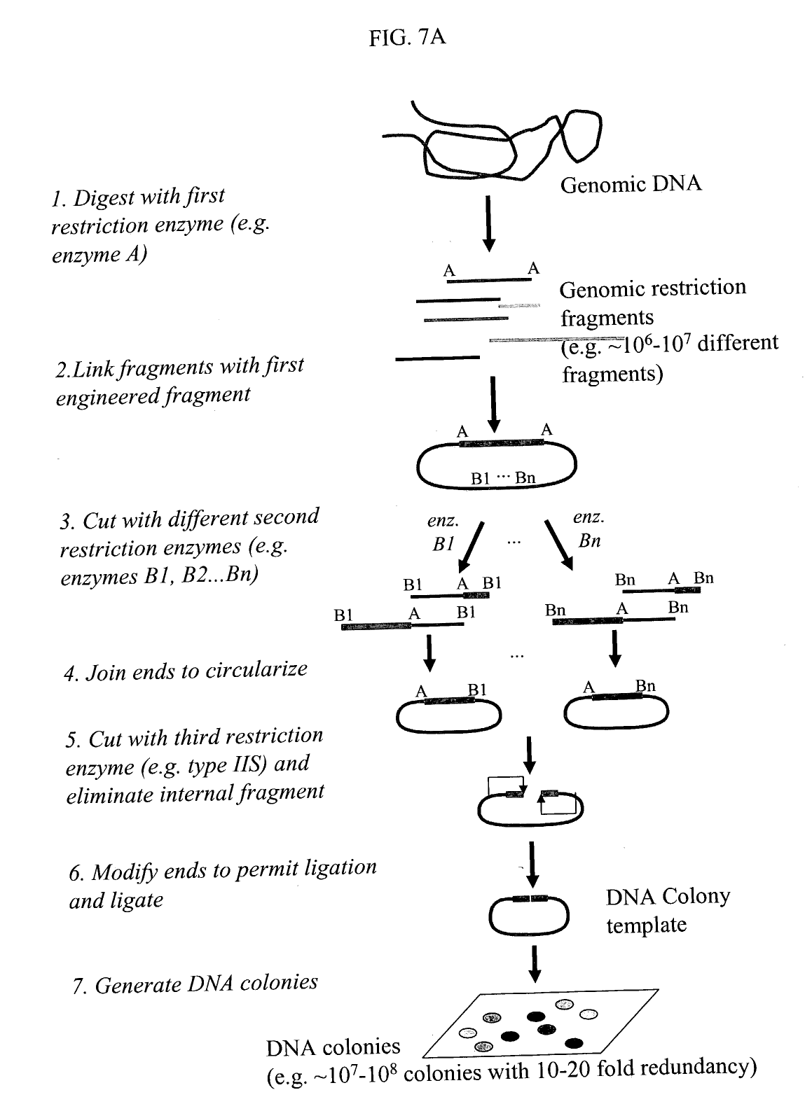

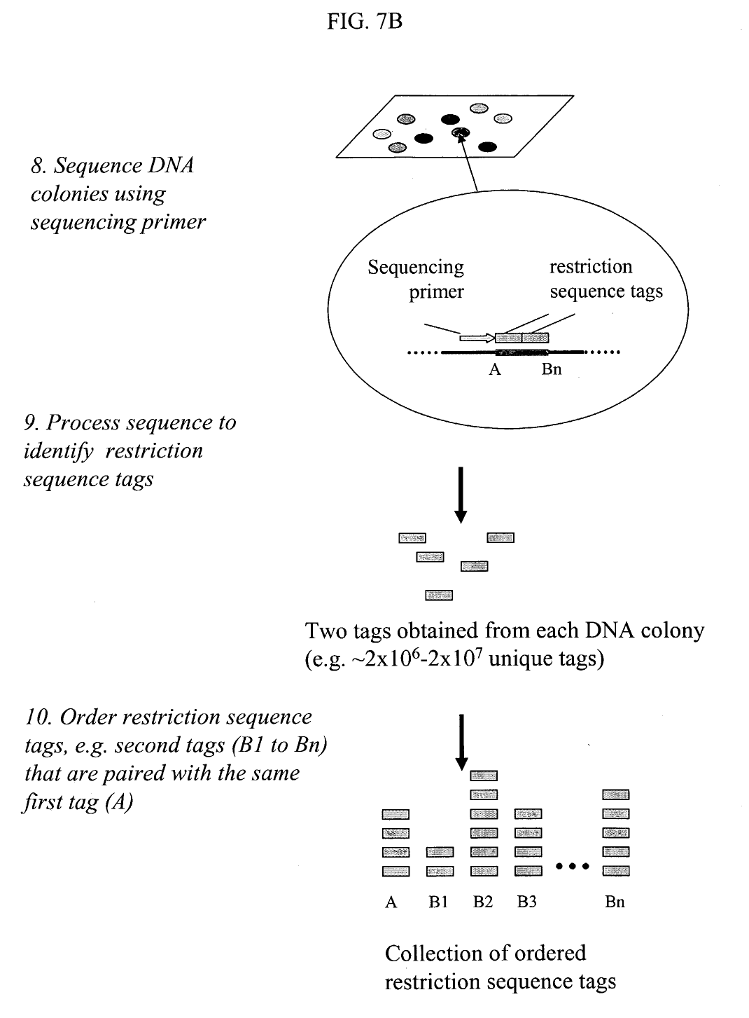

In another preferred embodiment (FIGS. 7A and 7B), after digestion by a first restriction enzyme, a set of restriction sequence tags is determined from the set of restriction fragments by a method comprising the step of:

1) linking the restriction fragments in the set of restriction fragments with a first engineered nucleic acid to obtain a set of first circular nucleic acid fragments, the first engineered nucleic acid comprising a predetermined nucleotide sequence comprising a recognition site of a second restriction enzyme and two recognition sites of a third restriction enzyme, the recognition site of the second restriction enzyme being located between the recognition sites of the third restriction enzyme, the recognition sites of the third restriction enzyme being located and oriented such that the third restriction enzyme cut in the restriction fragments, wherein the second restriction enzyme and the third restriction enzyme are different from each other;

2) digesting the first nucleic acid fragments with the second restriction enzyme to obtain a set of second nucleic acid fragments;

3) linking the ends of the second restriction fragments to produce a set of second circular nucleic acid fragments; and

4) sequencing at least a portion of each of the restriction fragments in the third circular nucleic acid fragments to determine the set of restriction sequence tags. Preferably, the method further comprises after the step 3) the steps of 3i) digesting the second circular nucleic acid fragments with the third restriction enzyme to produce a set of third nucleic acid fragments; 3ii) modifying the ends generated by the third restriction enzyme to permit ligation; and; and 3iii) linking the ends of the third nucleic acid fragments to produce a set of third circular nucleic acid fragments. Preferably, the recognition sites of the third restriction enzyme in the first engineered nucleic acid is located close to an end of the first engineered nucleic acid. In one preferred embodiment, each of the recognition sites of the third restriction enzyme in the first engineered nucleic acid is located less than 20 nucleotides from an end of the first engineered nucleic acid. In a more preferred embodiment, each of the recognition sites of the third restriction enzyme in the first engineered nucleic acid is located zero to 5 nucleotides from an end of the first engineered nucleic acid. Preferably, the third restriction enzyme is a type IIs endonuclease. In a preferred embodiment, the type IIs endonuclease cuts more than 5, 10, 20, 50, 100, or more than 200 bases from its recognition site.

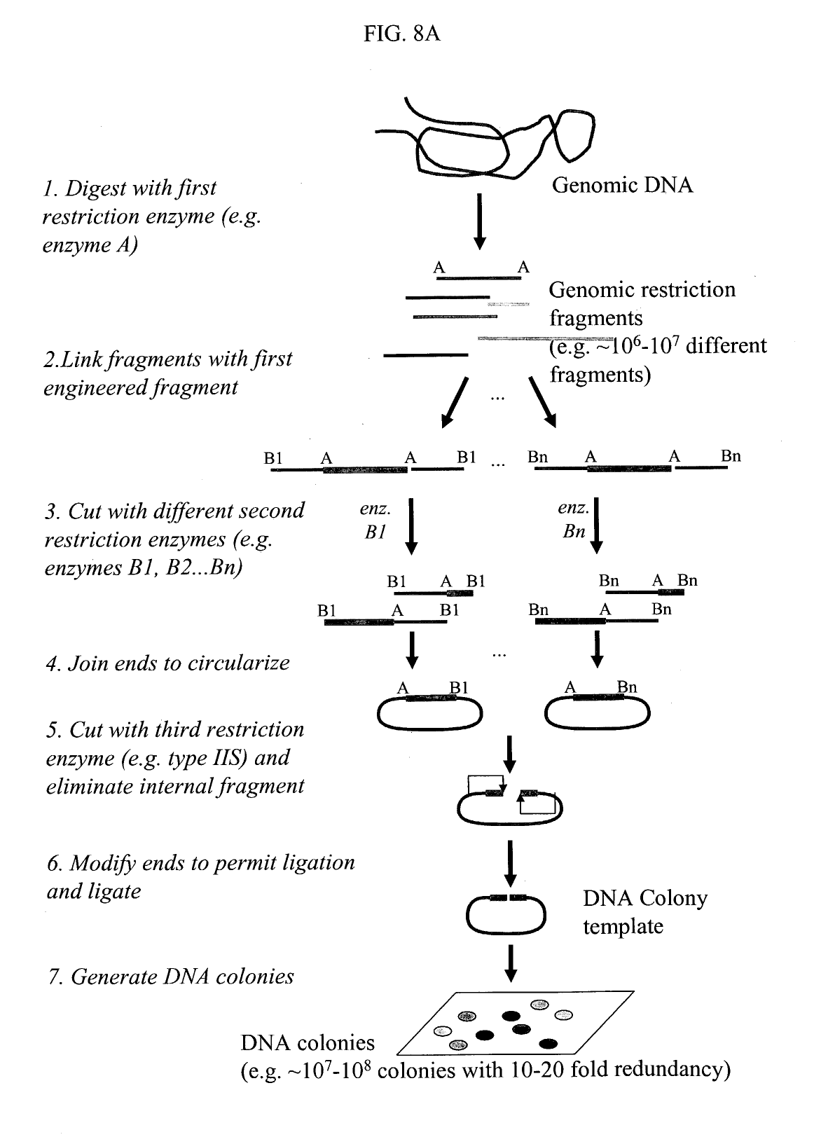

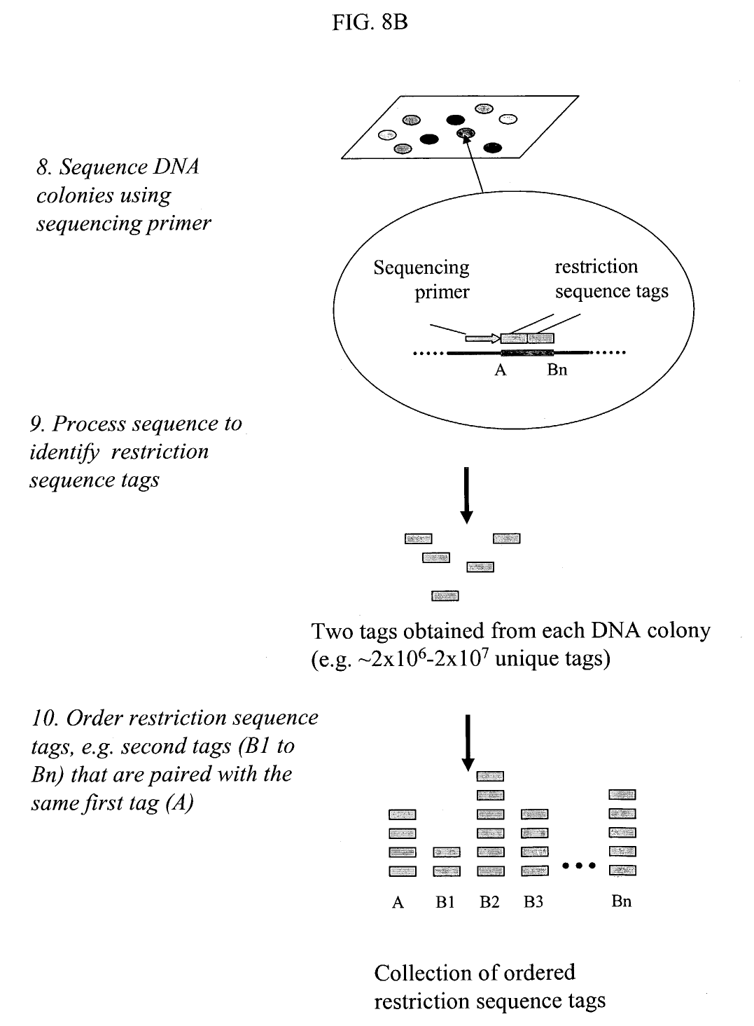

In still another preferred embodiment (FIGS. 8A and 8B), after digestion by a first restriction enzyme, a set of restriction sequence tags is determined from the set of restriction fragments by a method comprising the step of:

1) linking the restriction fragments in the set of restriction fragments with a first engineered nucleic acid to obtain a set of first nucleic acid fragments, the first engineered nucleic acid comprising a predetermined nucleotide sequence comprising a recognition site of a second restriction enzyme different from the first restriction enzyme;

2) digesting the first nucleic acid fragments with the second restriction enzyme to obtain a set of second nucleic acid fragments;

3) linking the ends of the second restriction fragments to produce a set of first circular nucleic acid fragments; and

4) sequencing at least a portion of each of the fourth nucleic acid fragments, thereby determining the set of restriction sequence tags. Preferably, the method further comprises after the step 3) the steps of 3i) digesting the first circular nucleic acid fragments with a third restriction enzyme to produce a set of third nucleic acid fragments, wherein the third restriction enzyme is different from the first and second restriction enzymes; 3ii) modifying the ends generated by said third restriction enzyme to permit ligation; and 3iii) linking the ends of the third nucleic acid fragments to produce a set of second circular nucleic acid fragments.

For such embodiments, it is preferable that the set of restriction fragments generated by the first restriction enzyme are further digested separately with each of a plurality of different second restriction enzymes. More preferably, the plurality of different second restriction enzymes comprises at least 3, 5, 10 or 20 different restriction enzymes.

In preferred embodiments, a step of fixing and amplifying the first circular nucleic acid fragments is carried out before the step of sequencing. In more preferred embodiments, the fixing and amplifying is carried out by any one of the DNA colony methods described Section 5.3. In still more preferred embodiments, the sequencing is carried out by a base by base primer extension method described Section 5.3.

In still other preferred embodiments of the invention, the step of modifying the ends of the second restriction fragments are done by fill-in the ends or removing the overhanging nucleotides of the second restriction fragments with a DNA polymerase such that the ends are blunt and can be linked.

In another preferred embodiment, the method of the invention comprises purification step and/or DNA isolation steps after each step.

Such embodiments permit identifying the two restriction sequence tags comprised in each first restriction fragment parts, wherein first restriction tag is next to first restriction enzyme recognition site and wherein second restriction tag is next to second restriction enzyme recognition site, and storing the information that the first and second restriction sequence tags are paired restriction sequence tags originated from the same first restriction fragment.

Restriction sequence tags can be grouped by means of sequence homology and, if possible, further grouping the paired restriction sequence tags containing the same first restriction sequence tag and storing the information that the second restriction tags from grouped paired restriction sequence tags are physically located close to—and on the same side of—a given first restriction enzyme recognition site. In preferred methods of the invention, if the genomic sequence is available, an additional step of clustering restriction sequence tags by means of mapping to identify flanking restriction sequence tags that are located on the genome on both sides of the recognition site of the first restriction enzyme is provided.

### 6. EXAMPLES

The following examples are presented by way of illustration of the present invention, and are not intended to limit the present invention in any way.

### 6.1. Example 1

Preparation of DNA Colonies Templates: Double Restriction Sequence Tag

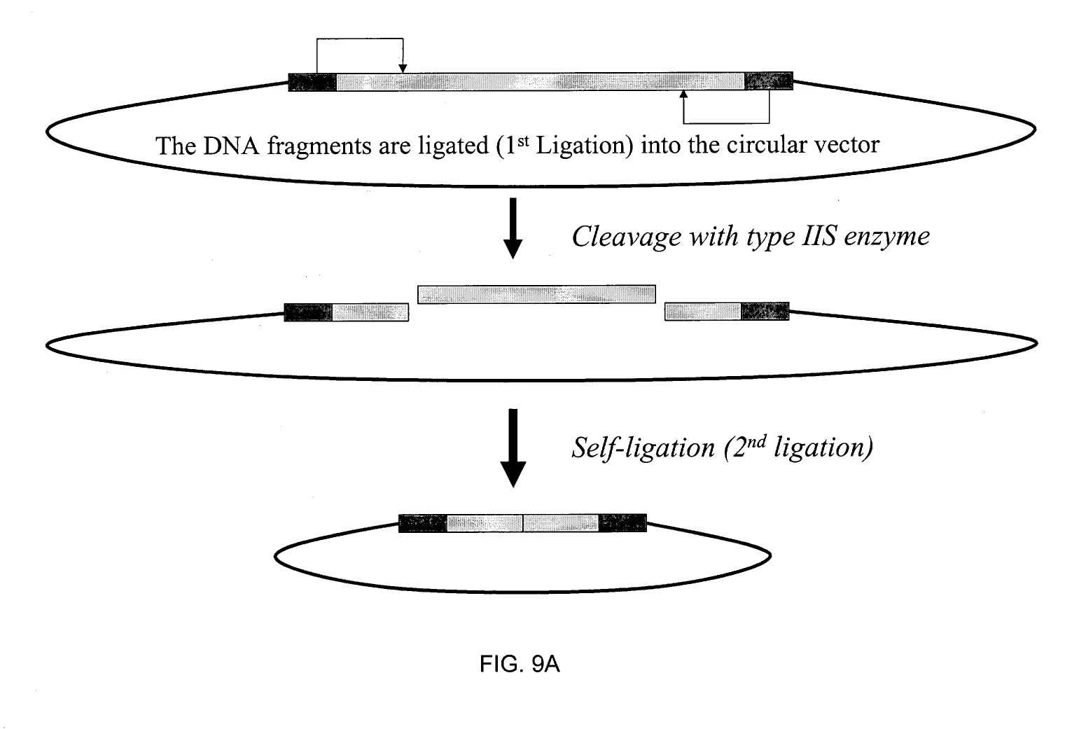

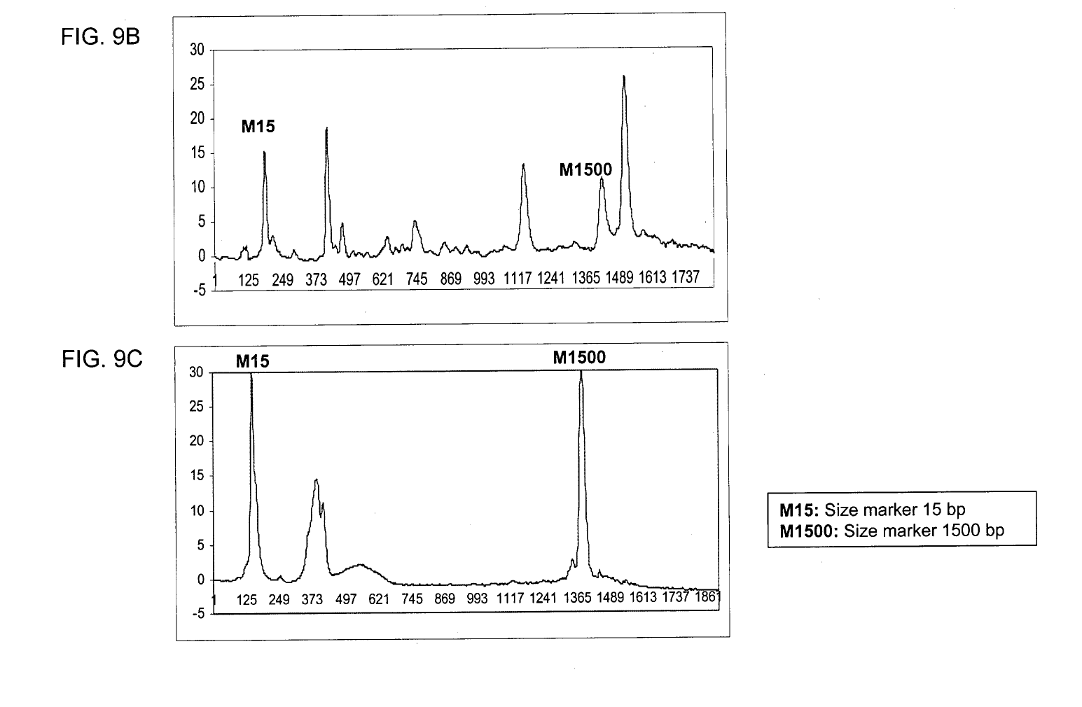

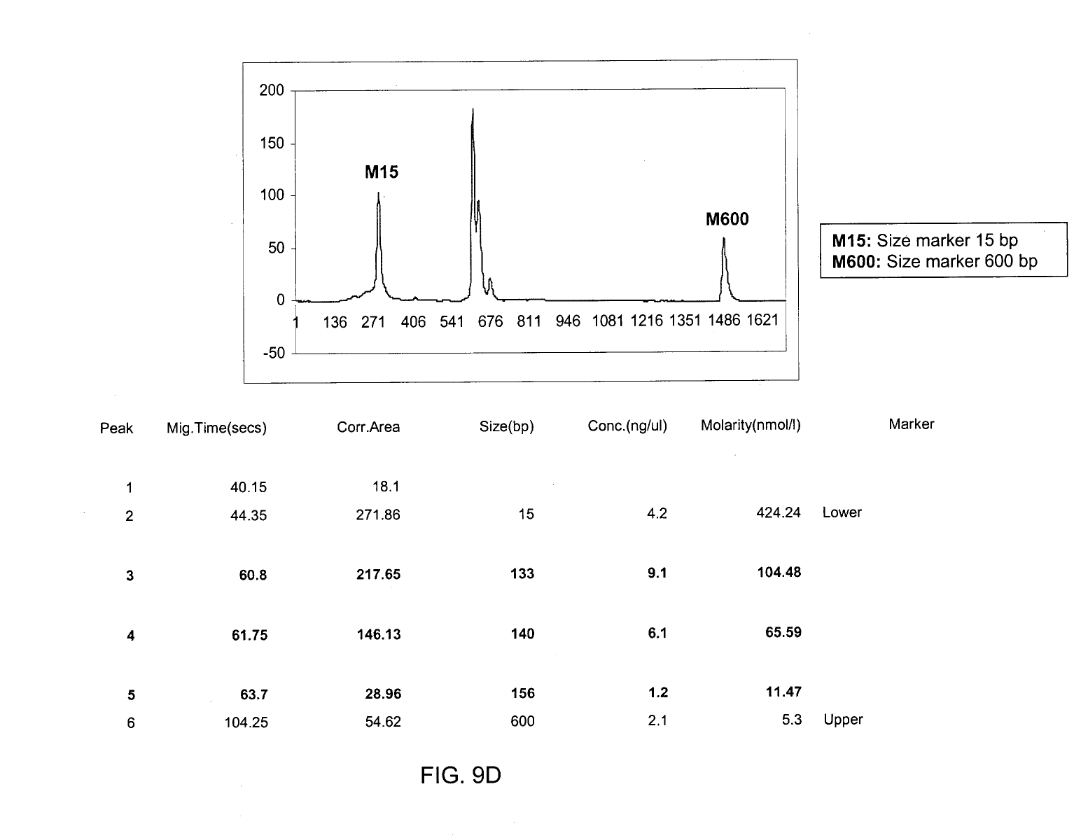

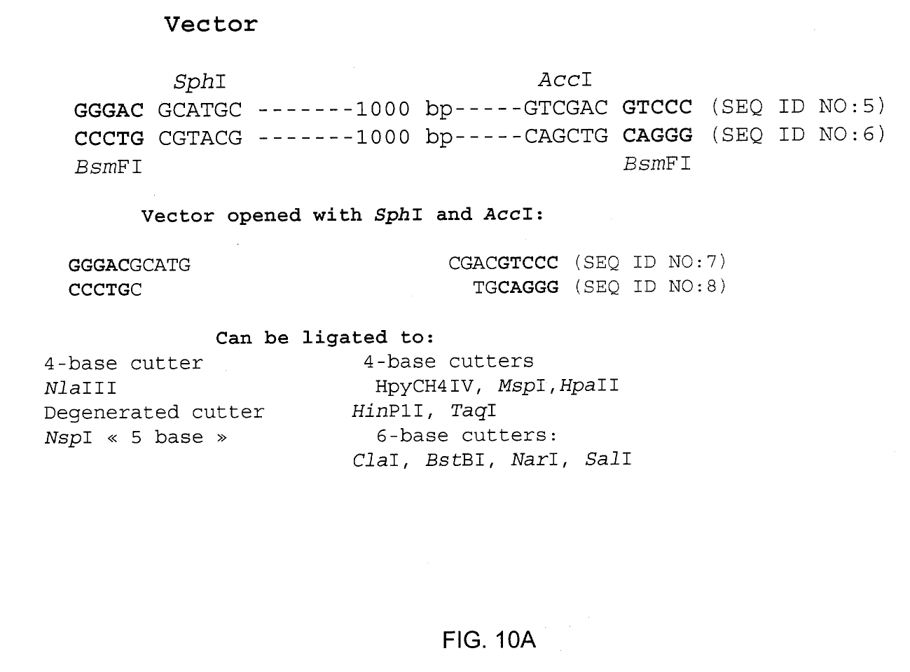

This example illustrates the engineering a vector for in vitro generation of DNA tags. An embodiment of generation of restriction sequence tags from genomic DNA is shown in FIG. 9A. This example utilized a plasmid vector carrying DNA cloning sites situated between two BsmFI sites. The vector is based on pUC19 plasmid, which was chosen due to its small size.

3) 1^{st} Generation of Cloning Vectors

A 1^{st} generation of cloning vectors were designed for use with genomic DNA digested with a single restriction enzyme. In this example, bacteriophage lambda genomic DNA was used to demonstrate the generation of restriction sequence tags.

Two variants of the vector were made by cloning synthetic linkers into pUC19. In the first variant, the vector contains an insert

| | |
|---|---|
| BsmFI BamHI BsmFI | |
| GGGAC GGATCC GTCCC | (SEQ ID NO:1) |
| CCCTG CCTAGG CAGGG | (SEQ ID NO:2) |

This allows the cloning of Sau3AI digested lambda DNA into the BamHI restriction site flanked by two BsmFI sites. The BamHI site of pUC19 was previously removed from the vector.

In the second variant, the vector contains an insert having an AatII restriction site (underlined) formed by two adjacent BsmFI sites:

| | |
|---|---|
| BsmFI BsmFI | |
| GGGAC GTCCC | (SEQ ID NO:3) |
| CCCTG CAGGG | (SEQ ID NO:4) |
| AatII | |

This allows the cloning of TaiI digested Lambda DNA into the vector. The AatII site of pUC19 was previously removed from the vector.

Both 1^{st} generation vectors were dephosphorylated prior to use in order to prevent self-ligation of the empty vector. After the ligation of lambda DNA fragments, DNA Polymerase I and ligase were used to restore the integrity of both DNA strands.

The following summarizes the steps (common also for further generations of vectors) used:

i) First ligation

ii) Inactivation of T4 DNA ligase by heating

iii) Digest with BsmFI

iv) Filling-in DNA ends by Klenow enzyme and dNTPs

v) Inactivation of BsmFI by heating

vi) Second ligation reaction

The following protocol of in vitro Restriction Sequence Tags generation was used in the example:

1^{st} Ligation

0.1 μg bacteriopahge lambda genomic DNA cleaved by appropriate enzymes

0.05 μg linear vector (purified by agarose gel)

1 mM ATP

1-x buffer NEB4

1 μl T4 DNA Ligase (New England Biolabs, 400 u/μl)

Total volume 10 μl, incubate 2 hours at room temperature

Inactivate T4 DNA ligase by heating 65° C. for 20 min

BsmFI Digest

Add 5 μl of solution containing:

1-x buffer NEB4

0.5 μl BsmF1 (New England Biolabs, 2 u/μl)

Incubate 2 hours at 65° C.

Klenow Treatment

Add 5 μl of solution containing:

1-x buffer for T4 DNA Ligase (New England Biolabs)

100 μM dNTPs

0.5 μl Klenow fragment (New England Biolabs, 5 u/μl)

Incubate 5 min at room temperature

Inactivate enzymes by heating 80° C. for 20 min

2^{nd} Ligation

Add the equal volume of solution containing

1-x MSL buffer

2 mM ATP

20% PEG6000

10% (v/v) T4 DNA Ligase (New England Biolabs, 400 u/μl)

Incubate over night at 16° C.

In the protocol above, a Minimal Salt Ligation (MSL) Buffer were used because intramolecular ligation is more efficient in low salt. The composition of MSL buffer is shown below:

| | |
|---|---|
| 5-x MSL | 1-x MSL |
| 50 mM Tris-HCl pH 7.5 | 10 mM Tris-HCl pH 7.5 |
| 50 mM MgCl₂ | 10 mM MgCl₂ |
| 10 mM DTT | 1 mM DTT |

The analysis of in-vitro ligation products was performed by PCR. An amplification product of 134 bp is formed if the two Lambda DNA restriction sequence tags of the correct size are present in the vector. Amplification products of smaller sizes can be formed by, e.g., insertion of only one tag into the vector, empty vector without any tag, or the BsmFI digest of empty vector followed by self-ligation.

The analysis of the length of PCR products was performed using Agilent DNA500 or DNA1000 chip. Another way of investigation of the in-vitro ligation products to transform them into competent E. coli cells followed by analysis of plasmids isolated from the individual colonies. When the products of first ligation of lambda DNA into the vector were analyzed, the multiple peaks were observed (FIG. 9B) as a result of insertion of DNA fragments of different lengths into the vector.

When products of the second ligation were analyzed, the fragment of expected size was present together with smaller fragments (FIG. 9C).

Although the 1^{st} generation vector permits size standardization of lambda genomic DNA into two Restriction Sequence Tags of the expected size, some undesired products were detected. The reason for it is probably self-ligation of vector during the first ligation reaction. This can occur as a result of uncompleted dephosphorylation or can be induced by DNA Polymerase I treatment, which is able to remove dephosphorylated bases from the vector ends. The problem can be overcome by partial filing of the genomic DNA fragment as illustrated in the example with a single Restriction Sequence Tag. For instance, the BamHI site can be partially filled with dGTP.

Alternatively, the vector can be designed by replacing the BamHI site with a BglII site. Ligation of the BamHI genomic fragments into the BglII digested vector in the presence of BglII restriction enzyme will prevent self-ligation of the vector. Only the expected vector-insert ligation product will suppress the BglII site and therefore resist digestion.

The BsmFI enzyme was evaluated in a simple construct. A circular plasmid which contains a 2000 bp DNA insert in the BamHI site of the 1^{st} generation vector was digested using BsmFI (no sites within the insert) and the 3000 bp band of the vector containing the attached DNA tags was isolated from agarose gel. This DNA was treated with Klenow enzyme+dNTPs to generate blunt ends and with T4 ligase for the 2^{nd} ligation. The results presented on FIG. 9D indicate the absence of bands of fragments smaller than the expected size of 133 bp. The extra bands of fragments of a larger size are likely to be PCR artifacts, because they were not observed in subsequent experiments.

This experiment indicates that BsmFI enzyme cleaves precisely at the correct distance and the generation of tags can be performed successfully. An alternative to the generation of blunt ends to permit ligation of the two Restriction Sequence tags is to insert a linker between the two restriction sequence tags.

Another option is to reverse the vector-insert-linker system. The first ligation links the genomic DNA fragment with the linker (containing the unique cutting site that will be useful for linearization of the DNA colonies and permit sequencing of both strands of the DNA amplified in each DNA colony). After digest with a type IIS enzyme, the “vector” arms are ligated to the ends cut by the type IIS enzyme.

2) The 2^{nd} Generation Vectors

A 2^{nd} generation vector was designed in order to use two different enzymes for cloning, e.g. to permit further reduction of the average size of the genomic DNA fragments and avoid self-ligation of the empty vector. To facilitate the separation of a fully cleaved plasmid from the partially digested one on agarose gel, a 1000 bp DNA fragment (derived from BlueScript plasmid pBSK) was included between the restriction sites of the raw vector. Dephosphorylation and DNA polymerase 1 treatment are not required for the 2^{nd} generation vector.

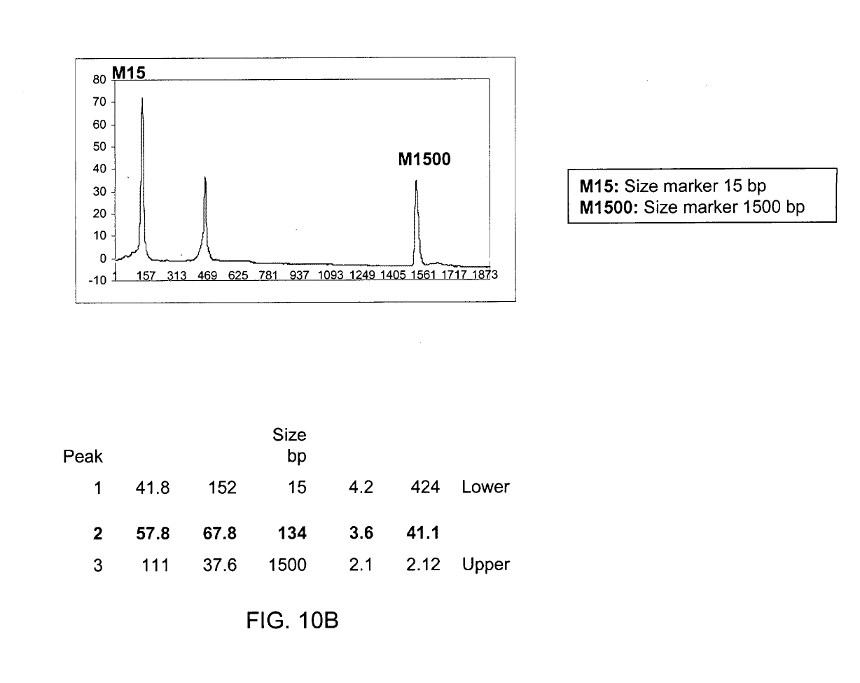

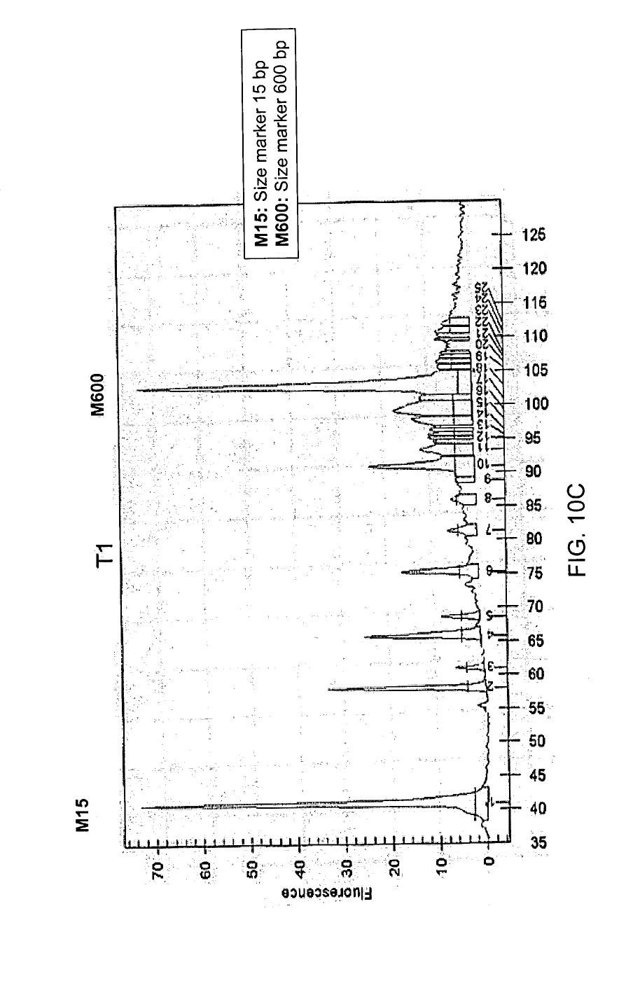

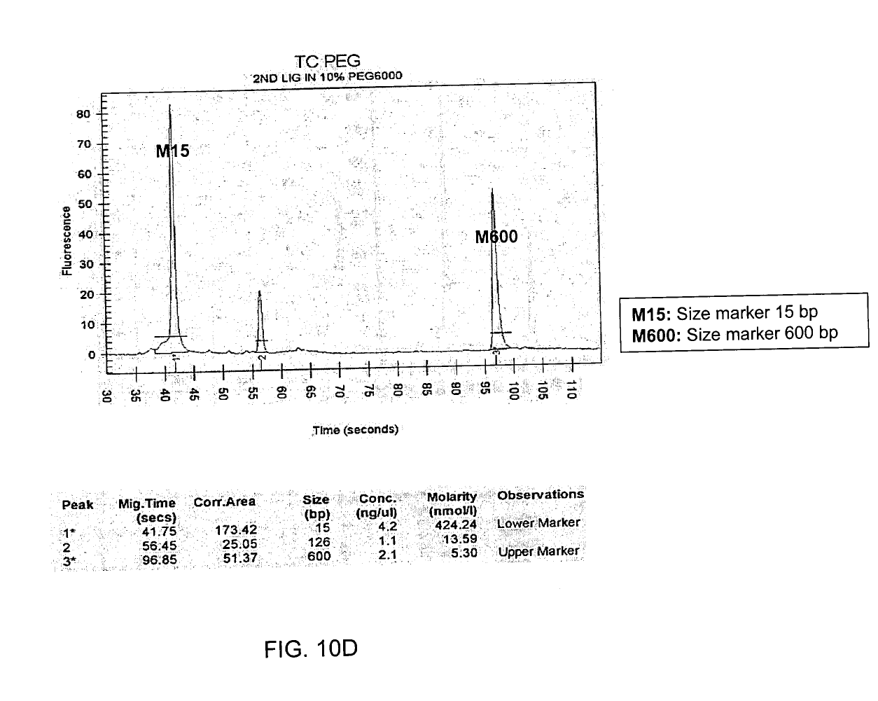

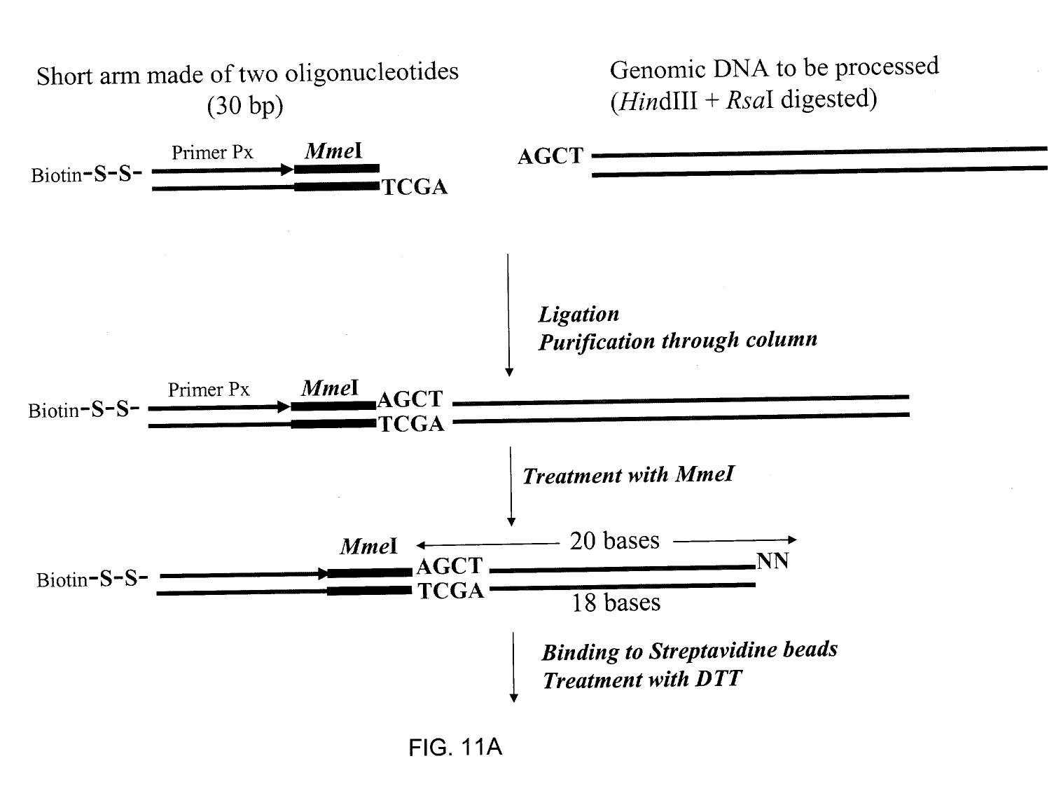

The raw vector contains an insert as shown in FIG. 10A, which allows the use of SphI and AccI restriction sites for cloning. The self SphI and AccI sites of pUC19 plasmid were removed. Due to the 3′ protruding end formed by SphI digestion, the empty vector cannot autoligate unless the Klenow enzyme completely removes the overhang. The DNA digested by two different enzymes can be inserted into 2^{nd} generation vector. FIG. 10A shows several possibilities of cloning.

The in vitro ligation of Lambda DNA digested with MspI and SphI into SphI-AccI opened vector was performed. The analysis of 2^{nd} ligation products indicated the presence of a single band of correct size, as shown in FIG. 10B. The products of the second ligation were transformed into E. coli cells. Thirty (30) colonies were inoculated into liquid cultures. Twelve (12) plasmids from bacterial cultures with highest density were analyzed. No plasmids corresponding to the empty vector were observed. No plasmids with insert size variation more than two bases were observed.

A similar experiment was carried out with AluI and SphI digested lambda DNA that was inserted into HincII and SphI digested vector (AluI and HincII generate blunt ends). FIG. 10C shows the results of analysis of products of the first ligation. Fragments of different sizes from lambda DNA were observed by analysis using Agilent 2100 bioanalyzer DNA 1000 chip, as expected. The highest peaks are the size markers. FIG. 10D shows the results of analysis of products of the second ligation. Only a single fragment of the expected size was observed by analysis using Agilent 2100 bioanalyzer DNA 1000 chip.

### 6.2. Example 2

Preparation of DNA Colonies Templates: Single Restriction Sequence Tag

This example illustrates the preparation of DNA colony templates each containing a single Restriction Sequence Tags from a DNA sample to be genotyped, as depicted in FIG. 4A. The size standardization step of this protocol ensures an efficient and comparable amplification of all DNA colonies, as the variable fragment, the Restriction Sequence Tag, represents less than 6% of the size of the DNA colony template. The insertion into the DNA colony vector permits the addition of universal sequences to generate DNA colony templates.

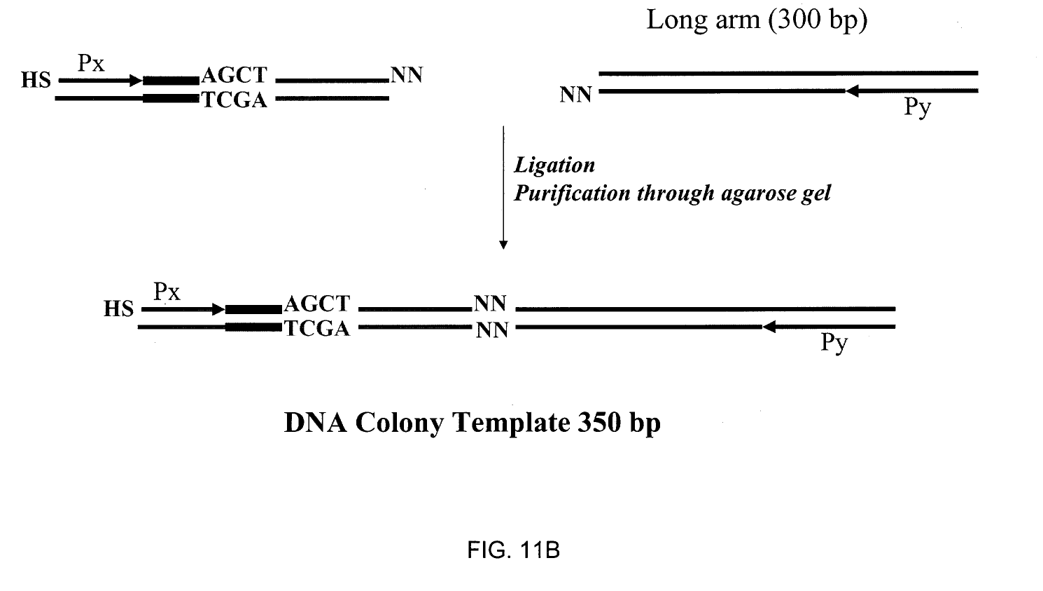

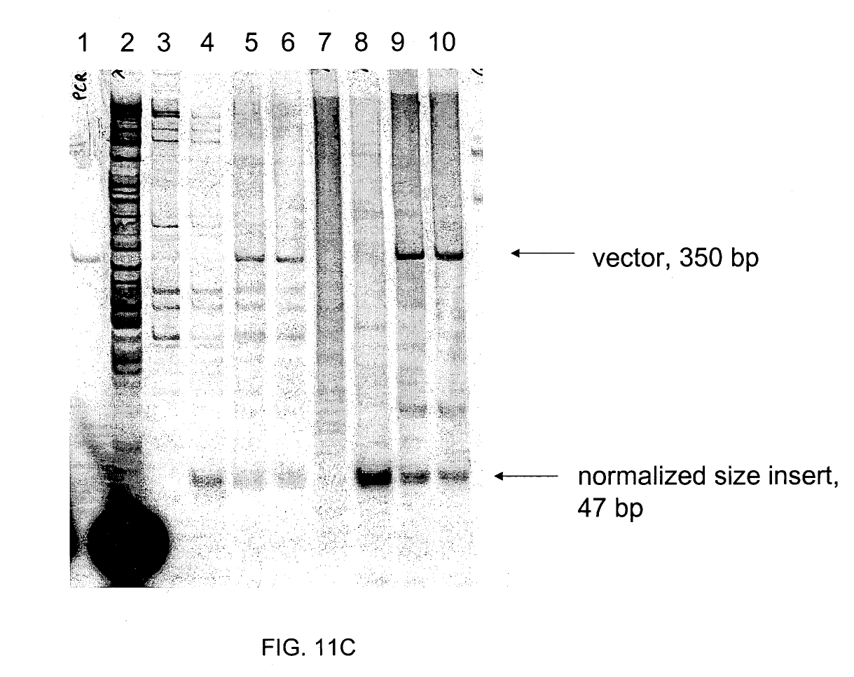

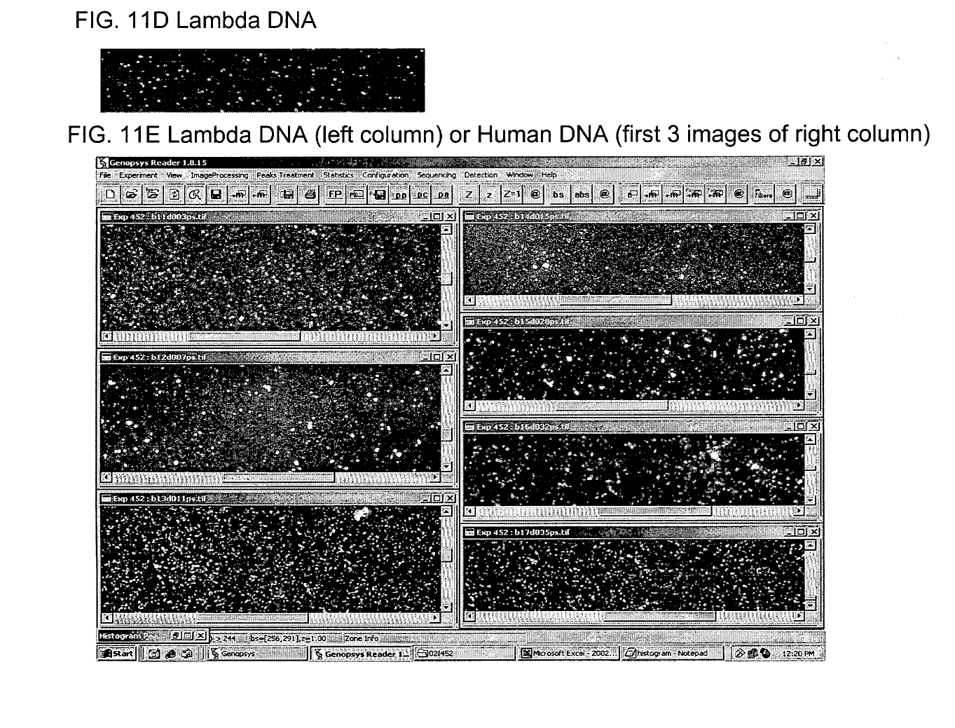

The general strategy of in vitro cloning used in this example is shown in FIGS. 11A-B. Briefly, the short double stranded adaptor (called “short arm”) consist of amplification primer Px followed by hexanucleotide TCCGAC forming the recognition site of the type IIs restriction enzyme MmeI. The 5′ end of the oligonucleotide contains a biotin moiety bound through a cleavable disulfide bond. The complementary strand is 5′-phosphorylated and contains extended nucleotides that are compatible with the sticky ends of DNA digested by the initial restriction enzyme. The short arm is ligated with DNA cleaved with a corresponding endonuclease and further treated with a type IIS enzyme MmeI. This leaves a 20 bp fragment of DNA attached to the short arm. The conjugate is then purified from other DNA fragments using streptavidin beads and ligated to the “long arm” containing another amplification primer Py.

Even when the cloning strategy is based upon the DNA cleavage by an endonuclease recognising a 6 bp sequence (HindIII), the digestion of DNA with a second frequently cutting enzyme (4 bp recognition site, RsaI) is preferable in order to reduce the average DNA fragment size.

The protocols for template preparation from the lambda phage DNA digested with HindIII and RsaI and the different generated steps are summarized below:

i) Digestion of lambda genomic DNA

ii) Ligation to the short Px arm

iii) Digestion by MmeI

iv) Purification of Px arm-tag conjugate

v) Attachment of Py arm

vi) Final DNA colony template purification

Protocol for each individual step used in this example is described in detailed below.

i) Digestion of Bacteriophage Lambda Genomic DNA

Lambda genomic DNA is digested with both HindIII and RsaI.

Mix 10 μl of lambda bacteriophage DNA (New England Biolabs, 0.5 μg/μl) with 5 μl of buffer Y+/Tango (Fermentas); 32.5 μl H₂O; 1.25 μl HindIII (New England Biolabs); 1.25 μl RsaI (New England Biolabs).

Incubate at 37° C. for 2 to 16 hours.

This gives 100 ng/μl solution of lambda phage DNA which contains 42 fmol/μl of HindIII ends.

Partial filling of the HindIII Overhangs with dATP

Different protocols can be used to maximise the ligation of the lambda genomic DNA HindIII ends with the short arm vector while preventing self-ligation of the lambda genomic fragments and self-ligation of the short arms.

It was discovered that the best method to prevent self-ligation of the HindIII ends is a step of single base filling with dATP. The short arm fragments must also be designed to be compatible with the partially filled HindIII ends of the genomic DNA fragments.

Filling the HindIII Ends:

Mix 20 μl of HindIII-RsaI digested lambda genomic DNA with 2 μl 10 mM dATP; 1 μl Klenow enzyme (New England Biolabs, 5 u/μl).

Incubate 30 min at 25° C. and 20 min at 70° C.

ii) Ligation to the Short Arm Moiety

Care should be taken to prevent the formation of short arm dimers (or to eliminate them from solution) during this ligation reaction. Such dimers, formed after partial MmeI digestion, may give rise to templates of correct size containing the cloned fragments of short arm.

As indicate above, the use of short arms containing non-palindromic overhangs complementary to partially filled DNA end is the preferred method. Alternatively, short arms containing a dideoxy base on its 3′ end may be used. MmeI can cleave the DNA if a nick is present right after the recognition site. The use of unphosphorylated short arm is another option.

This cloning step is performed by using 10 times molar excess of short arms over HindIII ends filled with dATP.

Preparation of the Short Arm:

Mix 10 μl of 10 μM solution of biotinylated oligo Short-A 5′-GAGGAAAGGG AAGGGAAAGG AAGGTCCGAC-3′ (SEQ ID NO: 9) in 10 mM Tris-HCl pH 8.0 with 10 μl of 10 μM solution of oligo Short-B 5′-GCTGTCGGAC CTTCCTTTCC CTTCCCTTTC CTC-3′ (SEQ ID NO: 10) in 10 mM Tris-HCl pH 8.0. Oligo Short-A contains a cleavable disulfide bridge between the biotin and its 5′ end.

Warm up to 80° C. and slowly cool to room temperature during 30 min.

Ligation:

To the partially filled HindIII ends of the genomic DNA mix, add 3 μl 10 mM riboATP; 4 μl of 5 μM short arm; 1 μl T4 DNA Ligase (New England Biolabs, 400 u/μL) and incubate for 1 hour at 16° C.

Proceed with DNA purification according to Qiagen MiniElute Reaction Clean Up protocol. Elute with 12 μl of buffer EB and repeat the elution without changing the tube using 5 μl fresh buffer EB.

Under these ligation conditions, there is no significant polymerisation of the genomic DNA fragments due to the ligation of the blunt RsaI-generated ends.

The purification of samples using Qiagen Mini Elute column instead of thermal inactivation of the T4 ligase is preferred in order to remove the majority of unligated arms. Double elution may increase the recovery of reaction products.

iii) MmeI Digestion

The effective digestion by MmeI is a critical step determining the template yield. The enzyme should be used with a ratio not more than 1-2 units per μg of DNA. According to New England Biolabs, excess of enzyme blocks the endonuclease cleavage.

To the sample mix, add 2 μl buffer Y+/Tango (Fermentas); 2 μl 1 mM SAM; 1 μl (2 u.) MmeI (New England Biolabs).

Incubate 37° C. for 1 hour.

iv) Binding/Release to the Streptavidin Beads.

Even though the manufacturer information indicates that a 30 min time is sufficient for binding of DNA to beads, overnight incubation strongly increases the yield of the product. The disulfide bond cleavage by 200 mM DTT and release of DNA is completed in 30 min. After this step, it is useful to analyze the yield of the desired product (50 bp) and the efficiency of MmeI digestion. Undigested products are seen as large DNA fragments.

Binding/Release to the SA Beads:

Add 10 μl of washed SA 280 beads (Dynal) resuspended in 20 μl of 2× B&W Buffer (made according to Dynal protocol).

Incubate overnight at room temperature with agitation. Wash the beads 2 times with 40 μl of 1× B&W buffer. Wash the beads 2 times with 40 μl 100 mM Tris-HCl pH 8.0. Add to the beads 11 μl of 200 mM DTT in 80 mM Tris-HCl pH 8.0.

Incubate 30 min at RT with agitation.

Separate the supernatant from beads and discard the beads. If necessary, analyze 1 μl of supernatant on Agilent 2100 bioanalyzer DNA 1000 chip.

v) Ligation of the Long Arm Moiety

This ligation is based on the recognition of the random two bases present in the a 3′-overhang generated in the genomic DNA by the MmeI ligation. As these two bases are degenerated, such ligation is a slow reaction and requires increased concentration of enzyme (New England Biolabs, information note about MmeI).

Preparation of the Long Arm:

To a tube with ready-to-use PCR beads (Amersham) add 19 μl of H₂O; 1 μl of 1 ng/μl pUC19 plasmid DNA (region 571-870 will be amplified); 2.5 μl of 10 μM oligo Long-A 5′-CTCACATTAA TTGCGTTGCG NNCACTGCCC GCTTTCCAG-3′ (SEQ ID NO: 11); 2.5 μl of 10 μM oligo Long-B 5′-CACCAACCCA AACCAACCCA AACCGAAAAA CGCCAGCAAC G-3′ (SEQ ID NO: 12). Perform amplification using program in PTC-200 thermocycler (MJ Research): 94° C. 2 min 30 sec; 25 cycles of (94° C. 30 sec ; 55° C. 30 sec; 72° C. 30 sec); followed by 72° C. 10 min.

The expected length of amplification product is 323 bp. The reaction product should be purified through Qiagen column and its purity and concentration estimated by analysis on Agilent 2100 bioanalyzer DNA 1000 chip.

The PCR product must then be digested BtsI. The amount of enzyme and incubation time depends on the amount of the PCR product. The efficiency of digestion should be estimated by analysis on Agilent 2100 bioanalyzer DNA 1000 chip. The change in size from 323 to 301 bp is expected. If digestion is complete, purification through Qiagen columns (PCR products purification protocol) is sufficient to remove the small 22 bp product from the reaction. Otherwise the 301 bp fragment should be purified through a 2% agarose gel.

Ligation of Long Arm Moiety:

To the supernatant released from the beads, add 2 μl of 100 mM MgCl₂; 2 μl of 10 mM rATP; 5 μl of long arm; 1 μl of concentrated T4 DNA Ligase (New England Biolabs, 2000 U/μl).

Incubate at 16° C. over night.

If necessary, analyse 1 μl of reaction on Agilent 2100 bioanalyzer DNA 1000 chip.

vi) Final Template Purification

For the final purification step, the desired ligated template is separated from free long arms and eventual long arm dimers or unreacted 50 bp products. The heating of the template in denaturing conditions should be avoided in order to minimize dissociation of the template strands.

Final Purification of Template:

Load the entire sample on 2% agarose gel. Run as long as good separation between free long arm (301 bp) template (350 bp) and the long arm dimer (600 bp) is achieved. Cut the band from agarose.

Purify DNA by Clontech Montage Agarose kit or Qiagen MiniElute Agarose Extraction Kit. If Qiagen Kit is used, do not warm up the tube at 50° C. as recommended, it will be dissolved at room temperature for 15 min. The final product can be analyzed on Agilent 2100 bioanalyzer DNA 1000 chip if necessary.

Size standardization was then verified. The same experiment was carried out in parallel starting from bacteriophage lambda genomic DNA or human genomic DNA that was labelled with ³³P-dATP.

FIG. 11 C shows aliquots collected after the various steps of the process and analysed by autoradiography. Lane 1: PCR product of complete DNA colony vector size, 350 bp; lane 2-6: lambda genomic DNA and lane 7-10 human genomic DNA; lane 3 and 7 after ligation to the short arm; lane 4 and 8 after digest with MmeI, the size standardization is observed; lane 5, 6, 9 and 10: after ligation with the long arm thus generating the DNA colony vector with expected size.

DNA colonies were then generated as follows: the DNA colony vectors, containing lambda or human genomic DNA fragments digested with HindIII and size standardized with MmeI, constructed as indicated in this example were used to generate DNA colonies using the method of WO 00/18957. FIG. 11D shows DNA colonies of Lambda DNA. FIG. 11E shows DNA colonies of Lambda DNA (left column) or Human DNA (first 3 images of right column). These DNA colonies are then sequenced in situ using the method of WO 98/44152 to identify the Restriction Sequence Tags.

The size of the DNA colony vector was also verified by PCR amplification. The PCR products were then cloned into the pUC19 plasmid and transformed in E. Coli competent cells (XL-2 Blue, Stratagene). Minipreps from individual clones were sequenced. It was verified that the Restriction Sequence Tags are of the expected size of 20 bp. However, tags of 21 bases long were recovered for some clones. No tags less than 20 bases were found.

A fingerprinting experiment demonstrated that all the expected 14 HindIII-digested lambda were present in the DNA Colony vectors. After the MmeI treatment and ligation of the long arm, the fragments were purified from an agarose gel and primer extension was carried out in presence of 3 dXTP and one dideoxy nucleotide (e.g. dATP, dTTP, dCTP and ddGTP). The products were then analyzed on an acrylamide gel permitting identification of each expected fragment.

If a 6 base cutter that generates 4 base overhangs is used for cloning, information about 21 consecutive bases can be obtained from the prepared templates. Out of 21, six are the known bases forming recognition site of endonuclease and 15 can be used for genetic variation detection. For some enzymes, this number can be increased if the “sticky” end of the short arm overlaps with the MmeI site, for example, TCCGA ligated to NcoI end CATGG forms MmeI site.

Two variations of the standard protocols described above were also used to increase the power of the cloning. In one variation, a blunt end-generating enzyme was used. If the enzyme used for DNA cleavage has a 6 base recognition sequence and leaves blunt ends, information about 23 consecutive bases (6 known and 17 for SNP detection) can be obtained. As the efficiency of blunt ended ligation is lower, extended ligation times are required. Nevertheless, sufficient ligation efficiency was achieved when ligation was performed overnight. The yield of the template obtained using MscI-digested lambda DNA was similar to the yield with HindIII digested lambda DNA.

The analysis of plasmids obtained by insertion of the amplified template into the pUC19 plasmid revealed the following: (1) the absence of “templates” containing short arm dimers; (2) low amount of undesired products (only 1-2 of 18 clones); (3) a good representation of the different lambda genomic DNA fragments in templates (only 3 fragments were found twice in a total of 15 templates).

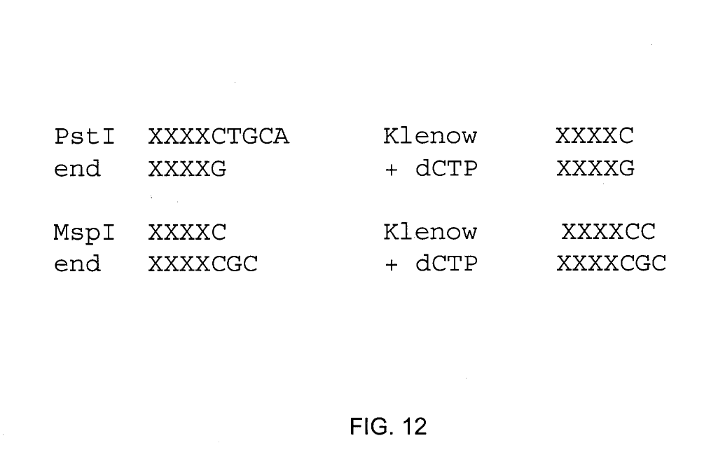

In another variation artificial generation of blunt ends was employed. If the 4 base overhangs that remain after the initial DNA digestion are removed, the cloning information increases to 25 bases (6 known and 19 for SNP detection). In preliminary experiments, two enzymes able of removing overhangs were investigated. Mung Bean nuclease (New England Biolabs) failed to generate blunt ends efficiently. The removal of 3′ overhangs by the Klenow enzyme yielded satisfactory results. Moreover, in the presence of necessary deoxynucleotide, the latter enzyme can also efficiently prevent the ends generated by frequent cutter from participating to the ligation. For example, if the DNA is digested by PstI and MspI, in the presence of dCTP the Klenow enzyme will polish PstI end and convert MspI end into inactive single base 5′ overhang (FIG. 12).

### 6.3. Example 3

Detection of Genome-Wide Sequence Variations

This example illustrates an embodiment of the invention which is used for generation of a high number of restriction sequence tags from a complex genome in a reproducible manner. These restriction sequence tags are useful for identifying genetic variants between genomes without prior knowledge of these variants and for identifying in a comprehensive manner and without hypothesis based on prior knowledge the variants associated with a phenotype specific to a population of individuals, and for correlating such variants, due to the high density of the restriction sequence tags obtained, to genomic regions of minimal sizes.

The method disclosed in this example is based on the use of the same restriction endonuclease to generate identical restriction fragments from different genomic DNA samples. After amplification, the ends of these restriction fragments are sequenced and the sequences are processed to identify restriction sequence tags, which are short sequence of nucleotides immediately next to the recognition site of the restriction enzyme used for digestion of the genomic DNA.

For each individual of the population under study, illustrated here by patients of a clinical study, this method is performed according to the following steps:

1) Extraction of Genomic DNA

Genomic DNA is extracted from biological samples from different individuals. These biological samples are either buccal swabs or blood samples. The genomic DNA is extracted using standard protocols. Typically, 0.5 to 3 micrograms of genomic DNA is extracted from a buccal swab sample and 4 micrograms of genomic DNA is extracted from 100 microliters of a whole blood sample. Since one diploid human genome has approximately 6 picograms of DNA, this corresponds to from at least 80 to over 600 copies of a diploid genome, which is sufficient for our purpose.

2) Restriction Digest.

The restriction endonuclease to be used is chosen according to the density of the restriction sequence tags in the genome that is to be obtained, which depends directly on the average distance between two restriction enzyme recognition sites (which is equivalent to the average length of genomic restriction fragments that will be obtained). Therefore, since the objective is to obtain on average at least one cut per every 5000 bases, a restriction enzyme with a 6 bases recognition site is used, as it is expected to generate fragments of average size of 4096 bases. Thus over 1,400,000 genomic restriction fragments for each diploid human genome which has approximately 6 billion bases are generated. Since for each genomic restriction fragment two restriction sequence tags are generated, an estimated total of over 2.8 million different restriction sequence tags are generated for a diploid human genome. As discussed below, restriction sequence tags generated in these examples are 15 bases long and that polymorphisms are found every 500 bases in the human genome, 2.8 million tags are estimated to generate over 80,000 polymorphisms per patient or one polymorphism every 35,000 bases of the human genome sequence.

The number of restriction sequence tags obtained per individual can be modulated by using different restriction enzymes or combinations of enzymes. For instance to increase the number of restriction sequence tags, a plurality of restriction enzymes can be used in combination or this method can be repeated sequentially with different enzymes. Alternatively, to decrease the number of restriction sequence tags, enzymes with longer recognition sites can be used, alone or in combination.

When the method is repeated on the same or different samples, it is essential that identical restriction fragments are generated, with the exception of variations due to changes in the genomic sequence between samples, so that equivalent restriction sequence tags will be obtained. In theory, different restriction enzymes with identical recognition sites, such as isoschizomers may be used. In these examples, however, identical enzymes, originating from the same organism and from the same supplier are used.

The restriction digest is carried out using at least 10 to 20 copies of the diploid genome per patient, a redundancy introduced to ensure that each restriction sequence tag will be represented.

3) Insertion of Genomic Restriction Fragments into Amplification and Sequencing Vectors

In this example, DNA colonies are used for amplification of the genomic restriction fragments and for sequencing.

The genomic restriction fragments are linked to a DNA colony vector, i.e., an engineered nucleic acid having a predetermined sequence, by performing a ligation reaction resulting in circular molecules. The DNA colony vector contains the following characteristics: two ends that are compatible with the ends of the digested genomic DNA fragments and preferably cohesive, which ends are dephosphorylated to prevent self-ligation of the vector; two recognition sites for a type IIS restriction enzyme, such as BsmFI, BceA1, Eco57I or MmeI, each of which is located immediately at an end and oriented to direct cut within the genomic restriction fragments to be linked with the vector; a recognition site for two sequencing primers, each of which is also close to an end of the vector and oriented to permit primer extension in the direction of the genomic restriction fragment to be linked with the vector; two amplification primers oriented to permit amplification of part of the vector and the inserted fragment, which may overlap with the sequence of the sequencing primers; and, optionally, a recognition site of a rare cutting restriction enzyme being located outside the region that will be amplified using the amplification primer sequence. Additional features of the DNA colony vector include additional restriction sites within the amplified region, e.g. for DNA colony linearization, or spacer sequences.

To prevent concatemerization of the genomic restriction fragments, DNA colony vector molecules are used in molar excess compared to the genomic restriction fragments.

4) Standardization of the Insert Size

The circular DNA molecules containing the DNA colony vectors linked to genomic restriction fragments are then digested with the type-IIs restriction enzyme. For instance if BceAI is used, it will cut 14 bases within the inserted genomic fragment. After a fill-in reaction with a DNA polymerase such as Klenow fragment of DNA polymerase I or T4 DNA polymerase, the resulting blunt ends are ligated resulting in circular molecules containing a 28 bases portion of a linked genomic restriction fragment, i.e., one 14 bases portion from each end of a genomic restriction fragment.

Longer inserts is generated using enzymes such as MmeI that cut 20 bases outside of its recognition sequence. However, due to the fact that a 2-base 3′ overhang is generated, the reaction with a DNA polymerase such as Klenow fragment of DNA polymerase I or T4 DNA polymerase will remove 2 bases. In this case, the resulting linked genomic restriction fragments are 36 bases long.

5) Generation of DNA Colony Templates

The DNA colony templates are generated using one or more cycles of PCR amplification in the presence of the amplification primers. A DNA template molecule sequence contains, from 5′ to 3′ end the following: a sequence of the first amplification primer in forward orientation; a sequence of the first sequencing primer in forward orientation (which can overlaps the sequence of the first amplification primer); a first recognition site of a type-IIS restriction enzyme; the 28 or 36 bases linked genomic restriction fragments resulting from the size standardization step (which includes half the recognition sites of the restriction enzyme used to digest the genomic DNA); a second recognition site of the type-IIS restriction enzyme; a sequence of the second sequencing primer in reverse orientation (which can overlap with the sequence of the second amplification primer sequence); and a sequence of the second amplification primer in reverse orientation.

Alternatively, DNA colony templates can be generated by simple restriction digest of the circular molecules obtained at previous step using the rare cutting enzyme that cuts the DNA colony vector outside the region to be amplified by the amplification primers.

6) Generation of DNA Colonies

The first step for generation of DNA colonies is to attach the DNA colony template molecules and the amplification primers on a solid surface, such as a surface of a functionalized glass or plastic such as NucleoLink tubes (Nunc, Roskilde, DK). The concentrations of the DNA colony templates and the amplification primer molecules are chosen such that after attachment, the surface is covered by a high density of amplification primer molecules and a relatively low density of DNA colony template molecules to permit localized amplification of the DNA colony template molecules into DNA colonies using the attached amplification primers and to achieve a desired spacing between different DNA colonies. The total number of DNA colonies after amplification should be at least 10 to 20 fold the number of different restriction fragments obtained from the genomic DNA to ensure appropriate redundancy. In the example in which 1.4 million genomic restriction fragments are generated, about 30 million DNA colonies are generated on a 3 square centimeters surface.

The amplification is carried out using the isothermal procedure (as described in Section 5.3 and PCT publication WO 02/46456).

7) Sequencing the DNA Colonies

After amplification, the DNA colonies are rendered single-stranded by restriction digest followed by denaturation. The first sequencing primer is then hybridized to the DNA colony vectors. The surface is then incubated with a mixture of DNA polymerase such as T7 DNA polymerase and only one of the 4 possible nucleotides. The mixture contains both fluorescently labeled and unlabelled nucleotide of the same kind so that approximately one in ten incorporated nucleotides is fluorescently labeled. These labeled nucleotides are incorporated at the 3′ end of the primer, if they are complementary to the sequence of the molecules in a DNA colony. After the primer extension step an image is taken by fluorescence microscopy (Axiovert 200, Zeiss, Germany, equipped with ORCA-ER CCD camera, Hamamatsu, Japan) to measure the position and intensity of the fluorescence of each DNA colony. This procedure is repeated in a stepwise fashion by repeatedly cycling through all 4 different kinds of nucleotides one after another. At each step, a given base is used for incorporation and the resulting signal is measured for each DNA colony on the surface. The fluorescence intensity of a DNA colony that has incorporated one or more the bases in the step become proportionately more intense, whereas that of a colony that does not incorporate the base remains unchanged. By comparing the fluorescence intensity after the step of incorporation to the intensity before the step, the amount of bases that have been incorporated in a DNA colony is determined. By following the sequential changes in fluorescence intensity for each DNA Colony and correlating the intensity with the identity of the base used for the extension step, the sequence of the DNA contained in each DNA colony is determined.

The sequencing steps are repeated until the 28 or 36 bases from the genomic fragment are read. The number of bases to be sequenced can be reduced by using a sequencing primer that extends to the half recognition site of the restriction enzyme used for the digestion of the genomic DNA.

If necessary, the extended first sequencing primer can be removed by denaturation and washing and sequencing of the complementary strand can be carried out using the second sequencing primer.

8) Restriction Sequence Tags

The sequences obtained from sequencing the DNA colonies are processed to identify the 2 restriction sequence tags from each original genomic restriction fragment. For instance, when the enzyme MmeI is used for standardization of the size of the linked restriction fragments, the restriction sequence tags are 18 bases long, minus the 3 bases from half of the restriction site used for digestion of the genomic DNA. With BceAI, the restriction sequence tags are 11 bases long.

These 2 restriction sequence tags represent the ends of the original genomic restriction fragment. The 2 tags obtained on each DNA colony are physically close on the genome (e.g. on average 4096 bases apart) and are stored for further use. The location of a tag on the genome is determined using the sequences consisting of the 15 or 11 bases plus the 6 bases of the restriction site of the restriction enzyme used for digestion of the genomic DNA, i.e., a 21 or 17 bases sequence.

9) Ordering the Restriction Sequence Tags and Identifying Sequence Variations Associated with a Phenotype

The restriction sequence tags are then compared using computer programs to identify the different tags and determine the number of each restriction sequence tag for each individual. These tags are then compared between individuals to identify groups of homologous tags and the sequence variations associated with a particular phenotype in the population. The comparisons can be carried out by statistical analysis known in the art, such as hidden Markov chains or a clustering method. The tags can also be compared with tags previously obtained or with sequences from databases.

Comparisons of restriction sequence tags between two populations can lead to different results. For a given sequence variation, the proportion of two types of genetic variants in population 1 can be different from the proportion in population 2.

In other instances the proportion of various types of sequence variations may be similar or identical in the two populations, but analysis of particular combinations of different genetic variants in individuals from each population can reveal that some combination of variants are represented in different proportions in the two populations.

Examples of groups of tags that could be obtained by the method of the invention:

In individual 1 it is determined

(SEQ ID NO:13)

T1a = acgtgtcgatggctgatgggtaggtagt, found 23 times

(SEQ ID NO:14)

T1b = ggtggtgggaatgggattggaaatgttt , found 11 times

(SEQ ID NO:15)

T1c = ggtggtgggaatcggattggaaatgttt , found 8 times

(SEQ ID NO:16)

T1e = ccaaggtgatcggatgtaatggtattgt , found 13 times

(SEQ ID NO:17)

T1f = ccaaggtgatcggaagtaatggtattgt , found 5 times

In individual 2 it is determined

(SEQ ID NO:13)

T2a = acgtgtcgatggctgatgggtaggtagt, found 18 times

(SEQ ID NO:14)

T2b = ggtggtgggaatgggattggaaatgttt, found 22 times

(SEQ ID NO:16)

T2c = ccaaggtgatcggatgtaatggtattgt, found 15 times

In individual 3 it is determined

(SEQ ID NO:13)

T3a = acgtgtcgatggctgatgggtaggtagt, found 20 times

(SEQ ID NO:15)

T3b = ggtggtgggaatcggattggaaatgttt, found 24 times

(SEQ ID NO:17)

T3c = ccaaggtgatcggaagtaatggtattgt, found 17 times

It can be determined that

Tags T1a, T2a and T3a are identical, and form group g1 of group-sequence Sg1=T1a

Tags T1b and T2b are identical, and form group g2 of group-sequence Sg2=T2b

Tags T1c and T3b are identical and form group g3 of group-sequence Sg3=T1c

Tags T1e and T2c are identical and form group g4 of group-sequence Sg4=T1e

Tags T1f and T3c are identical and form group g5 of group-sequence Sg5=T1f

It can be seen that

Sg2=ggtggtgggaat g ggattggaaatgttt (SEQ ID NO: 14)

Sg3=ggtggtgggaat c ggattggaaatgttt (SEQ ID NO: 15)

are identical up to one single base, but each of them is very different from Sg1, Sg4 and Sg5, and

Sg4=ccaaggtgatcgga t gtaatggtattgt (SEQ ID NO: 16)

Sg5=ccaaggtgatcgga a gtaatggtattgt (SEQ ID NO: 17)

are identical up to one single base, but each of them is very different from Sg1, Sg2 and Sg3.

Group G1 formed by Sg2 and Sg3, group G2 formed by Sg4 and Sg5, and group G3 formed by group Sg1 can then be created.

Because each individual carries two different sets of chromosomes, it can be seen that

(1) individual 1 carries 2 copies of Sg1, one copy of Sg2, one copy of Sg3, one copy of Sg4 and one copy of Sg5

(2) individual 2 carries two copies of Sg1, two copies of Sg2 and two copies of Sg4

(3) individual 3 carries two copies of Sg1, two copies of Sg3 and two copies of Sg5

Typical Result 1

In population 1 it is found

1000 copies of sequence tags Sg1

327 copies of sequence tags Sg2

673 copies of sequence tags Sg3

521 copies of sequence tags Sg4

479 copies of sequence tags Sg5

In population 2 it is found

1000 copies of sequence tags Sg1

345 copies of sequence tags Sg2

665 copies of sequence tags Sg3

502 copies of sequence tags Sg4

498 copies of sequence tags Sg5

Since there is no significant difference between the respective composition of groups G1, G2 and G3 between population 1 and population 2, it can be concluded that these groups are not associated with the phenotypic difference between the populations

Typical Result 2

In population 1 it is found

1000 copies of sequence tags Sg1

993 copies of sequence tags Sg2

7 copies of sequence tags Sg3

521 copies of sequence tags Sg4

479 copies of sequence tags Sg5

In population 2 it is found

1000 copies of sequence tags Sg1

946 copies of sequence tags Sg2

54 copies of sequence tags Sg3

502 copies of sequence tags Sg4

498 copies of sequence tags Sg5

Since there is no significant difference between the respective composition of groups G1 and G3 between population 1 and population 2, it can be concluded that these groups are not associated with the phenotypic difference between the populations. Since there is a significant difference in the composition of group G2 between population 1 and population 2, it can be concluded that this group is associated with the phenotypic difference between the populations. Further, it can be concluded that the probability of belonging to population 2 is higher for individuals who carry sequence Sg3 than for individuals who carry Sg2.

Typical Result 3

In population 1 it is found

1000 copies of sequence tags Sg1

314 copies of sequence tags Sg2

686 copies of sequence tags Sg3

486 copies of sequence tags Sg4

514 copies of sequence tags Sg5

In population 2 it is found

1000 copies of sequence tags Sg1

289 copies of sequence tags Sg2

711 copies of sequence tags Sg3

511 copies of sequence tags Sg4

489 copies of sequence tags Sg5

There is no significant difference between the respective composition of groups G1, G2 and G3 between population 1 and population 2. However, further analysis of the data can be carried out by counting how many individuals carry the combinations of sequence tags:

| | population 1 | population 2 |
|---|---|---|
| Sg2,Sg3,Sg4,Sg5 | 109 | 143 |
| Sg2,Sg3,Sg4 | 44 | 26 |
| Sg3,Sg3,Sg5 | 59 | 62 |
| Sg2,Sg4,Sg5 | 26 | 8 |
| Sg3,Sg4,Sg5 | 115 | 104 |
| Sg2,Sg4 | 11 | 1 |
| Sg2,Sg5 | 14 | 20 |
| Sg3,Sg4 | 63 | 101 |
| Sg3,Sg5 | 59 | 35 |

This analysis shows a significant difference in the distribution of combinations of sequence tags between population 1 and population 2. It can thus be concluded that these combinations of sequence tags are associated with the phenotypic difference between the populations.

7. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of the present invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method for determining genome-wide sequence variations associated with a phenotype of one or more individual organisms, comprising
I) generating a set of restriction sequence tags for each individual organism of said one or more individual organisms by a method comprising
A) digesting nucleic acids from each said individual organism using one or more first restriction enzymes to generate a set of restriction fragments; and
B) determining a set of restriction sequence tags for each said individual organism, wherein said set of restriction sequence tags comprises one or more restriction sequence tags for each of said restriction fragments, each said one or more restriction sequence tags comprising a sequence in the corresponding restriction fragment; and
II) grouping restriction sequence tags for said one or more individual organisms into one or more groups of restriction sequence tags, each said group comprising restriction sequence tags that are homologous;
wherein said one or more groups of restriction sequence tags identify sequence variations associated with said phenotype.

2. The method of claim 1, wherein said step of determining said set of restriction sequence tags is carried out by a method comprising
B1) linking restriction fragments in said set of restriction fragments with a first engineered nucleic acid to obtain a set of first circular nucleic acid fragments, said first engineered nucleic acid comprising a predetermined nucleotide sequence comprising one or more recognition sites of a second restriction enzyme, said recognition sites being located and oriented such that said second restriction enzyme cut in said restriction fragments;
B2) digesting said first circular nucleic acid fragments with said second restriction enzyme;
B3) modifying the ends generated by said second restriction enzyme to permit ligation;
B4) linking said ends generated by said second restriction enzyme to produce a set of second circular nucleic acid fragments; and
B5) sequencing at least a portion of each of said restriction fragments in said second circular nucleic acids to determine said set of restriction sequence tags.

3. The method of any one of claims 2, wherein each said one or more recognition sites are located close to an end of said first engineered nucleic acid.

4. The method of claim 3, wherein each of said one of more recognition sites is located less than 25 nucleotides apart from an end of said first engineered nucleic acid.

5. The method of claim 4, wherein each of said one or more recognition sites is located zero to 5 nucleotides apart from an end of said first engineered nucleic acid.

6. The method of claim 2, wherein said second restriction enzyme is a type IIS endonuclease.

7. The method of claim 6, further comprising before said step B5) a step of fixing and amplifying nucleic acid fragments comprised in said second circular nucleic acid fragments on a solid surface.

8. The method of claim 7, wherein said step of fixing and amplifying is carried out by generating colonies of said nucleic acid fragments in said second circular nucleic acid fragments on said solid surface, wherein each of said colonies comprises a plurality of immobilized single stranded DNA molecules comprising one of said nucleic acid fragments in said second circular nucleic acid fragments.

9. The method of claim 8, wherein said colonies are generated by a method comprising
i) linearizing said second circular nucleic acid fragments to generate linearized fragments;
ii) providing a solid surface comprising a plurality of colony primers immobilized on said solid surface at 5′ end, wherein each said colony primer comprises a sequence that is hybridizable to a sequence at the 3′ end of said linearized fragments;
iii) denaturing said linearized fragments to generate single stranded fragments;
iv) annealing said single stranded fragments to said immobilized colony primers;
v) carrying out a primer extension reaction using said annealed single stranded fragments as templates to generate immobilized double stranded nucleic acid fragments;
vi) denaturing said immobilized double stranded nucleic acid fragments to generate immobilized single stranded fragments;
vii) annealing said immobilized single stranded fragments to immobilized colony primers;
viii) repeating said steps v) through vii) such that said colonies are generated, each at a particular location on said solid surface.

10. The method of claim 8, wherein said colonies are generated by a method comprising
i) linearizing said second circular nucleic acid fragments to generate linearized fragments;
ii) mixing said linearized fragments with colony primers, wherein each said colony primer comprises a sequence that is hybridizable to a sequence at the 3′ end of said linerarized fragments;
iii) grafting said linearized fragments and colony primers on a solid surface at the 5′ end to generate immobilized linearized fragments and immobilized colony primers;
iv) denaturing said immobilized linearized fragments to generate immobilized single-stranded fragments;
v) annealing said immobilized single stranded fragments to immobilized colony primers to obtain annealed single-stranded fragments;
vi) carrying out primer extension reaction using said annealed single stranded fragments as templates to generate immobilized double stranded nucleic acid fragments;
vii) denaturing said immobilized double stranded nucleic acid fragments to generate immobilized single stranded fragments;
viii) annealing said immobilized single stranded fragments to immobilized colony primers; and
ix) repeating said steps v) through viii) such that said colonies are generated, each at a particular location on said solid surface.

11. The method of claim 8, wherein said colonies are generated by a method comprising
i) linearizing said second circular nucleic acid fragments to generate linearized fragments;
ii) mixing said linearized fragments with colony primers, wherein each said colony primers comprises a sequence that is hybridizable to a sequence at the 3′ end of said linerarized fragments, and wherein the concentration of said colony primers is adjusted such that amplification of grafted linearized fragments can occur;
iii) grafting said linearized fragments and colony primers on a solid surface at the 5′ end to generate immobilized linearized fragments and immobilized colony primers;
iv) applying an amplification solution containing a polymerase and nucleotides to said solid surface such that said colonies are generated isothermally, each at a particularly location on said solid surface.

12. The method of any one of claims 9-11, wherein said sequencing is carried out by a method comprising
i) hybridizing sequencing primers to said colonies;
ii) carrying out primer extension with one labeled nucleotide;
iii) detecting the amount of the labeled nucleotide which is incorporated into extended primers for each said location; and
iv) repeating steps ii) and iii) to determine a portion of the nucleotide sequence of each of said colonies.

13. The method of claim 12, wherein said labeled nucleotide is a fluorecently-labeled nucleotide, and wherein said detecting involves detecting the fluorescence intensity of said labeled nucleotide.

14. The method of claim 1, wherein said first restriction enzyme cuts at both sides of its recognition site in such a manner that the cutting sites enclose a part of sequence that is not part of the recognition site, and wherein said step of determining said set of restriction sequence tags is carried out by a method comprising
B1) modifying the ends generated by said first restriction enzyme to permit ligation;
B2) linking said restriction fragments in said set of restriction fragments with a first engineered nucleic acid to obtain a set of first circular nucleic acid fragments, said first engineered nucleic acid comprising a predetermined nucleotide sequence; and
B3) sequencing at least a portion of each of said restriction fragments in said first circular nucleic acids to determine said set of restriction sequence tags.

15. The method of claim 14, further comprising before said step B3) a step of fixing and amplifying nucleic acid fragments comprised in said second circular nucleic acid fragments on a solid surface.

16. The method of claim 15, wherein said step of fixing and amplifying is carried out by generating colonies of said nucleic acid fragments in said first circular nucleic acid fragments on said solid surface, wherein each of said colonies comprises a plurality of immobilized single stranded DNA molecules of one of said nucleic acid fragments in said first circular nucleic acid fragments.

17. The method of claim 16, wherein said colonies are generated by a method comprising
i) linearizing said first circular nucleic acid fragments to generate linearized fragments;
ii) providing a solid surface comprising a plurality of colony primers immobilized at the 5′ end on said solid surface, wherein each said colony primer comprises a sequence that is hybridizable to a sequence at the 3′ end of said linearized fragments;
iii) denaturing said linearized fragments to generate single stranded fragments;
iv) annealing said single stranded fragments to said immobilized colony primers;
v) carrying out primer extension reaction using said annealed single stranded fragments as templates to generate immobilized double stranded nucleic acid fragments;
vi) denaturing said immobilized double stranded nucleic acid fragments to generate immobilized single stranded fragments;
vii) annealing said immobilized single stranded fragments to immobilized colony primers;
viii) repeating said steps v) through vii) such that said colonies are generated, each at a particular location on said solid surface.

18. The method of claim 16, wherein said colonies are generated by a method comprising
i) linearizing said first circular nucleic acid fragments to generate linearized fragments;
ii) mixing said linearized fragments with colony primers, wherein each said colony primer comprises a sequence that is hybridizable to a sequence at the 3′ end of said linerarized fragments;
iii) grafting said linearized fragments and colony primers on a solid surface at the 5′ end to generate immobilized linearized fragments and immobilized colony primers;
iv) denaturing said immobilized linearized fragments to generate immobilized single-stranded fragments;
v) annealing said immobilized single stranded fragments to immobilized colony primers to obtain annealed single-stranded fragments;
vi) carrying out primer extension reaction using said annealed single stranded fragments as templates to generate immobilized double stranded nucleic acid fragments;
vii) denaturing said immobilized double stranded nucleic acid fragments to generate immobilized single stranded fragments;
viii) annealing said immobilized single stranded fragments to immobilized colony primers; and
ix) repeating said steps v) through viii) such that said colonies are generated, each at a particular location on said solid surface.

19. The method of claim 16, wherein said colonies are generated by a method comprising
i) linearizing said first circular nucleic acid fragments to generate linearized fragments;
ii) mixing said linearized fragments with colony primers, wherein each said colony primers comprises a sequence that is hybridizable to a sequence at the 3′ end of said linerarized fragments, and wherein the concentration of said colony primers is adjusted such that amplification of grafted linearized fragments can occur;
iii) grafting said linearized fragments and colony primers on a solid surface at the 5′ end to generate immobilized linearized fragments and immobilized colony primers;
iv) applying an amplification solution containing a polymerase and nucleotides to said solid surface such that said colonies are generated isothermally, each at a particularly location on said solid surface.

20. The method of any one of claims 17-19, wherein said sequencing is carried out by a method comprising
i) hybridizing sequencing primers to said colonies;
ii) carrying out primer extension with one labeled nucleotide;
iii) detecting the amount of the labeled nucleotide which is incorporated into extended primers for each said location; and
iv) repeating steps ii) and iii) to determine a portion of sequence of each of said colony.

21. The method of claim 20, wherein said labeled nucleotide is a fluorecently-labeled nucleotide, and wherein said detecting involves detecting the fluorescence intensity of said labeled nucleotide.

22. The method of claim 1, wherein said step of determining said set of restriction sequence tags is carried out by a method comprising
B1) linking said restriction fragments in said set of restriction fragments with a first engineered nucleic acid to obtain a set of first nucleic acid fragments, said first engineered nucleic acid comprising a predetermined nucleotide sequence comprising a recognition site of a second restriction enzyme, said recognition site being located and oriented such that said second restriction enzyme cut in said restriction fragments;
B2) digesting said first nucleic acid fragments with said second restriction enzyme;
B3) modifying the ends generated by said second restriction enzyme to permit ligation;
B4) linking said ends generated by said second restriction enzyme with a second engineered nucleic acid to produce second nucleic acid fragments, said second engineered nucleic acid comprising a predetermined nucleotide sequence; and
B5) sequencing at least a portion of each of said restriction fragments in said second nucleic acid fragments to determine said set of restriction sequence tags.

23. The method of claim 22, wherein said recognition site of said second restriction enzyme is located close to an end of said first engineered nucleic acid.

24. The method of claim 23, wherein said recognition site is located less than 25 nucleotides apart from said end of said first engineered nucleic acid.

25. The method of claim 24, wherein said recognition site is located zero to 5 nucleotides apart from said end of said first engineered nucleic acid.

26. The method of claim 22, wherein said second restriction enzyme is a type IIs endonuclease.

27. The method of claim 26, further comprising before said step B5) a step of fixing and amplifying nucleic acid fragments in said second nucleic acid fragments on a solid surface.

28. The method of claim 27, wherein said step of fixing and amplifying is carried out by generating colonies of said nucleic acid fragments in said second nucleic acid fragments on said solid surface, wherein each of said colonies comprises a plurality of immobilized single stranded DNA molecules of one of said nucleic acid fragments in said second nucleic acid fragments.

29. The method of claim 28, wherein said colonies are generated by a method comprising
i) providing a solid surface comprising a plurality of colony primers immobilized on said solid surface at 5′ end, wherein each said colony primer comprises a sequence that is hybridizable to a sequence at the 3′ end of said second nucleic acid fragments;
ii) denaturing said second nucleic acid fragments to generate single stranded fragments;
iii) annealing said single stranded fragments to said immobilized colony primers;
iv) carrying out primer extension reaction using said annealed single stranded fragments as templates to generate immobilized double stranded nucleic acid fragments;
v) denaturing said immobilized double stranded nucleic acid fragments to generate immobilized single stranded fragments;
vi) annealing said immobilized single stranded fragments to immobilized colony primers;
vii) repeating said steps iv) through vi) such that said colonies are generated, each at a particular location on said solid surface.

30. The method of claim 28, wherein said colonies are generated by a method comprising
i) mixing said second nucleic acid fragments with colony primers, wherein each said colony primers comprises a sequence that is hybridizable to a sequence at the 3′ end of said second nucleic acid fragments;
ii) grafting said second nucleic acid fragments and colony primers on a solid surface at the 5′ end to generate immobilized nucleic acid fragments and immobilized colony primers;
iii) denaturing said immobilized nucleic acid fragments to generate immobilized single-stranded fragments;
iv) annealing said immobilized single stranded fragments to immobilized colony primers to obtain annealed single-stranded fragments;
v) carrying out primer extension reaction using said annealed single stranded fragments as templates to generate immobilized double stranded nucleic acid fragments;
vi) denaturing said immobilized double stranded nucleic acid fragments to generate immobilized single stranded fragments;
vii) annealing said immobilized single stranded fragments to immobilized colony primers; and
viii) repeating said steps iv) through vii) such that said colonies are generated, each at a particular location on said solid surface.

31. The method of claim 28, wherein said colonies are generated by a method comprising
i) mixing said second nucleic acid fragments with colony primers, wherein each said colony primers comprises a sequence that is hybridizable to a sequence at the 3′ end of said second nucleic acid fragments, and wherein the concentration of said colony primers is adjusted such that amplification of grafted second nucleic acid fragments can occur;
iii) grafting said second nucleic acid fragments and colony primers on a solid surface at the 5′ end to generate immobilized second nucleic acid fragments and immobilized colony primers;
iv) applying an amplification solution containing a polymerase and nucleotides to said solid surface such that said colonies are generated isothermally, each at a particularly location on said solid surface.

32. The method of any one of claims 29-31, wherein said sequencing is carried out by a method comprising
i) hybridizing sequencing primers to said colonies;
ii) carrying out primer extension with one labeled nucleotide;
iii) detecting the amount of the labeled nucleotide which is incorporated into extended primers for each said location; and
iv) repeating steps ii) and iii) to determine a portion of sequence of each of said colony.

33. The method of claim 32, wherein said labeled nucleotide is a fluorecently-labeled nucleotide, and wherein said detecting involves detecting the fluorescence intensity of said labeled nucleotide.

34. The method of claim 1, wherein said first restriction enzyme is a rare cutter and wherein said step of determining said set of restriction sequence tags is carried out by a method comprising
B1) linking said restriction fragments in said set of restriction fragments with a first engineered nucleic acid to obtain a set of first nucleic acid fragments, said first engineered nucleic acid comprising a predetermined nucleotide sequence;
B2) digesting said first nucleic acid fragments with one or more second restriction enzymes to obtain second restriction fragments, wherein said second restriction enzymes are different from said first restriction enzyme and does not cut in said first engineered nucleic acid;
B3) linking the ends of said second restriction fragments with a second engineered nucleic acid to produce a set of second nucleic acid fragments, said second engineered nucleic acid comprising a predetermined nucleotide sequence; and
B4) sequencing at least a portion of each of said restriction fragments in said second nucleic acid fragments to determine said set of restriction sequence tags.

35. The method of claim 34, wherein said rare cutter recognizes a 6-base recognition sequence.

36. The method of claim 34, wherein said rare cutter recognizes an 8-base or a more than 8-base recognition sequence.

37. The method of claim 34, further comprising before said step B4) a step of fixing and amplifying nucleic acid fragments in said second nucleic acid fragments on a solid surface.

38. The method of claim 37, wherein said step of fixing and amplifying is carried out by generating colonies of said nucleic acid fragments in said second nucleic acid fragments on said solid surface, wherein each of said colonies comprises a plurality of immobilized single stranded DNA molecules of one of said nucleic acid fragments in said second nucleic acid fragments.

39. The method of claim 38, wherein said colonies are generated by a method comprising
i) providing a solid surface comprising a plurality of colony primers immobilized on said solid surface at 5′ end, wherein each said colony primer comprises a sequence that is hybridizable to a sequence at the 3′ end of said second nucleic acid fragments;
ii) denaturing said second nucleic acid fragments to generate single stranded fragments;
iii) annealing said single stranded fragments to said immobilized colony primers;
iv) carrying out primer extension reaction using said annealed single stranded fragments as templates to generate immobilized double stranded nucleic acid fragments;
v) denaturing said immobilized double stranded nucleic acid fragments to generate immobilized single stranded fragments;
vi) annealing said immobilized single stranded fragments to immobilized colony primers;
vii) repeating said steps iv) through vi) such that said colonies are generated, each at a particular location on said solid surface.

40. The method of claim 38, wherein said colonies are generated by a method comprising
i) mixing said second nucleic acid fragments with colony primers, wherein each said colony primers comprises a sequence that is hybridizable to a sequence at the 3′ end of said second nucleic acid fragments;
ii) grafting said second nucleic acid fragments and colony primers on a solid surface at the 5′ end to generate immobilized second nucleic acid fragments and immobilized colony primers;
iii) denaturing said immobilized second nucleic acid fragments to generate immobilized single-stranded fragments;
iv) annealing said immobilized single stranded fragments to immobilized colony primers to obtain annealed single-stranded fragments;
v) carrying out primer extension reaction using said annealed single stranded fragments as templates to generate immobilized double stranded nucleic acid fragments;
vi) denaturing said immobilized double stranded nucleic acid fragments to generate immobilized single stranded fragments;
vii) annealing said immobilized single stranded fragments to immobilized colony primers; and
iii) repeating said steps iv) through vii) such that said colonies are generated, each at a particular location on said solid surface.

41. The method of claim 38, wherein said colonies are generated by a method comprising
i) mixing said second nucleic acid fragments with colony primers, wherein each said colony primers comprises a sequence that is hybridizable to a sequence at the 3′ end of said second nucleic acid fragments, and wherein the concentration of said colony primers is adjusted such that amplification of grafted second nucleic acid fragments can occur;
iii) grafting said second nucleic acid fragments and colony primers on a solid surface at the 5′ end to generate immobilized second nucleic acid fragments and immobilized colony primers;
iv) applying an amplification solution containing a polymerase and nucleotides to said solid surface such that said colonies are generated isothermally, each at a particularly location on said solid surface.

42. The method of any one of claims 39-41, wherein said sequencing is carried out by method comprising
i) hybridizing sequencing primers to said colonies;
ii) carrying out primer extension with one labeled nucleotide;
iii) detecting the amount of the labeled nucleotide which is incorporated into extended primers for each said location; and
iv) repeating steps ii) and iii) to determine a portion of sequence of each of said colony.

43. The method of claim 42, wherein said labeled nucleotide is a fluorecently-labeled nucleotide, and wherein said detecting involves detecting the fluorescence intensity of said labeled nucleotide.

44. The method of claim 1, wherein said step of determining said set of restriction sequence tags is carried out by a method comprising
B1) linking said restriction fragments in said set of restriction fragments with a first engineered nucleic acid to obtain a set of first nucleic acid fragments, said first engineered nucleic acid comprising a predetermined nucleotide sequence;
B2) digesting said first nucleic acid fragments with a second restriction enzyme to obtain second restriction fragments, wherein said second restriction enzyme is different from said first restriction enzyme and does not cut in said first engineered nucleic acid;
B3) linking the ends of said second restriction fragments with a second engineered nucleic acid to produce a set of second nucleic acid fragments, said second engineered nucleic acid comprising a predetermined nucleotide sequence; and
B4) sequencing at least a portion of each of said restriction fragments in said second nucleic acid fragments to determine said set of restriction sequence tags.

45. The method of claim 44, further comprising repeating said steps B2) through B4) for each of a plurality of different second restriction enzymes.

46. The method of claim 45, further comprising before said step B5) a step of fixing and amplifying nucleic acid fragments in said second nucleic acid fragments on a solid surface.

47. The method of claim 46, wherein said step of fixing and amplifying is carried out by generating colonies of said nucleic acid fragments in said second nucleic acid fragments on said solid surface, wherein each of said colonies comprises a plurality of immobilized single stranded DNA molecules of one of said nucleic acid fragments in said second nucleic acid fragments.

48. The method of claim 47, wherein said colonies are generated by a method comprising
i) providing a solid surface comprising a plurality of colony primers immobilized on aid solid surface at 5′ end, wherein each said colony primer comprises a sequence that is hybridizable to a sequence at the 3′ end of said second nucleic acid fragments;
ii) denaturing said second nucleic acid fragments to generate single stranded fragments;
iii) annealing said single stranded fragments to said immobilized colony primers;
iv) carrying out primer extension reaction using said annealed single stranded fragments as templates to generate immobilized double stranded nucleic acid fragments;
v) denaturing said immobilized double stranded nucleic acid fragments to generate immobilized single stranded fragments;
vi) annealing said immobilized single stranded fragments to immobilized colony primers;
vii) repeating said steps iv) through vi) such that said colonies are generated, each at a particular location on said solid surface.

49. The method of claim 47, wherein said colonies are generated by a method comprising
i) mixing said second nucleic acid fragments with colony primers, wherein each said colony primers comprises a sequence that is hybridizable to a sequence at the 3′ end of said second nucleic acid fragments;
ii) grafting said second nucleic acid fragments and colony primers on a solid surface at the 5′ end to generate immobilized second nucleic acid fragments and immobilized colony primers;
iii) denaturing said immobilized second nucleic acid fragments to generate immobilized single-stranded fragments;
iv) annealing said immobilized single stranded fragments to immobilized colony primers to obtain annealed single-stranded fragments;
v) carrying out primer extension reaction using said annealed single stranded fragments as templates to generate immobilized double stranded nucleic acid fragments;
vi) denaturing said immobilized double stranded nucleic acid fragments to generate immobilized single stranded fragments;
vii) annealing said immobilized single stranded fragments to immobilized colony primers; and
viii) repeating said steps iv) through vii) such that said colonies are generated, each at a particular location on said solid surface.

50. The method of claim 47, wherein said colonies are generated by a method comprising
i) mixing said second nucleic acid fragments with colony primers, wherein each said colony primers comprises a sequence that is hybridizable to a sequence at the 3′ end of said second nucleic acid fragments, and wherein the concentration of said colony primers is adjusted such that amplification of grafted second nucleic acid fragments can occur;
iii) grafting said second nucleic acid fragments and colony primers on a solid surface at the 5′ end to generate immobilized second nucleic acid fragments and immobilized colony primers;
iv) applying an amplification solution containing a polymerase and nucleotides to said solid surface such that said colonies are generated isothermally, each at a particularly location on said solid surface.

51. The method of any one of claims 48-50, wherein said sequencing is carried out by a method comprising
i) hybridizing sequencing primers to said colonies;
ii) carrying out primer extension with one labeled nucleotide;
iii) detecting the amount of the labeled nucleotide which is incorporated into extended primers for each said location; and
iv) repeating steps ii) and iii) to determine a portion of sequence of each of said colony.

52. The method of claim 51, wherein said labeled nucleotide is a fluorecently-labeled nucleotide, and wherein said detecting involves detecting the fluorescence intensity of said labeled nucleotide.

53. The method of claim 1, wherein said step of determining said set of restriction sequence tags is carried out by a method comprising
B1) linking said restriction fragments in said set of restriction fragments with a first engineered nucleic acid to obtain a set of first circular nucleic acid fragments, said first engineered nucleic acid comprising a predetermined nucleotide sequence comprising a recognition site of a second restriction enzyme and two recognition sites of a third restriction enzyme, said recognition site of said second restriction enzyme being located between said two recognition sites of said third restriction enzyme, said recognition sites of said third restriction enzyme being located and oriented such that said third restriction enzyme cut in said restriction fragments, wherein said second restriction enzyme and said third restriction enzyme are different from each other;
B2) digesting said first nucleic acid fragments with said second restriction enzyme to obtain a set of second nucleic acid fragments;
B3) linking the ends of said second restriction fragments to produce a set of second circular nucleic acid fragments; and
B4) sequencing at least a portion of each of said restriction fragments in said third circular nucleic acid fragments to determine said set of restriction sequence tags.

54. The method of claim 53, further comprising after said step 3) the steps of
B5) digesting said second circular nucleic acid fragments with said third restriction enzyme to produce a set of third nucleic acid fragments;
B6) modifying the ends generated by said third restriction enzyme to permit ligation; and
B7) linking the ends of said third nucleic acid fragments to produce a set of third circular nucleic acid fragments.

55. The method of claim 53, further comprising repeating said steps B1) through B4) for each of a plurality of different second restriction enzymes.

56. The method of claim 55, wherein each of said recognition site is located close to an end of said first engineered nucleic acid.

57. The method of claim 56, wherein each of said recognition site is located less than 25 nucleotides apart from an end of said first engineered nucleic acid.

58. The method of claim 57, wherein each of said recognition site is located zero to 5 nucleotides apart from an end of said first engineered nucleic acid.

59. The method of claim 53, wherein said second restriction enzyme is a type IIs endonuclease.

60. The method of claim 59, further comprising before said step B4) a step of fixing and amplifying nucleic acid fragments in said second circular nucleic acid fragments on a solid surface.

61. The method of claim 60, wherein said step of fixing and amplifying is carried out by generating colonies of said nucleic acid fragments in said second circular nucleic acid fragments on said solid surface, wherein each of said colonies comprises a plurality of immobilized single stranded DNA molecules of one of said nucleic acid fragments in said second circular nucleic acid fragments.

62. The method of claim 61, wherein said colonies are generated by a method comprising
i) linearizing said second circular nucleic acid fragments to generate linearized fragments;
ii) providing a solid surface comprising a plurality of colony primers immobilized on said solid surface at 5′ end, wherein each said colony primer comprises a sequence that is hybridizable to a sequence at the 3′ end of said linearized fragments;
iii) denaturing said linearized fragments to generate single stranded fragments;
iv) annealing said single stranded fragments to said immobilized colony primers;
v) carrying out primer extension reaction using said annealed single stranded fragments as templates to generate immobilized double stranded nucleic acid fragments;
vi) denaturing said immobilized double stranded nucleic acid fragments to generate immobilized single stranded fragments;
vii) annealing said immobilized single stranded fragments to immobilized colony primers;
viii) repeating said steps v) through vii) such that said colonies are generated, each at a particular location on said solid surface.

63. The method of claim 61, wherein said colonies are generated by a method comprising
i) linearizing said second circular nucleic acid fragments to generate linearized fragments;
ii) mixing said linearized fragments with colony primers, wherein each said colony primers comprises a sequence that is hybridizable to a sequence at the 3′ end of said linerarized fragments;
iii) grafting said linearized fragments and colony primers on a solid surface at the 5′ end to generate immobilized linearized fragments and immobilized colony primers;
iv) denaturing said immobilized linearized fragments to generate immobilized single-stranded fragments;
v) annealing said immobilized single stranded fragments to immobilized colony primers to obtain annealed single-stranded fragments;
vi) carrying out primer extension reaction using said annealed single stranded fragments as templates to generate immobilized double stranded nucleic acid fragments;
vii) denaturing said immobilized double stranded nucleic acid fragments to generate immobilized single stranded fragments;
viii) annealing said immobilized single stranded fragments to immobilized colony primers; and
ix) repeating said steps v) through viii) such that said colonies are generated, each at a particular location on said solid surface.

64. The method of claim 61, wherein said colonies are generated by a method comprising
i) linearizing said second circular nucleic acid fragments to generate linearized fragments;
ii) mixing said linearized fragments with colony primers, wherein each said colony primers comprises a sequence that is hybridizable to a sequence at the 3′ end of said linerarized fragments, and wherein the concentration of said colony primers is adjusted such that amplification of grafted linearized fragments can occur;
iii) grafting said linearized fragments and colony primers on a solid surface at the 5′ end to generate immobilized linearized fragments and immobilized colony primers;
iv) applying an amplification solution containing a polymerase and nucleotides to said solid surface such that said colonies are generated isothermally, each at a particularly location on said solid surface.

65. The method of any one of claims 62-64, wherein said sequencing is carried out by a method comprising
i) hybridizing sequencing primers to said colonies;
ii) carrying out primer extension with one labeled nucleotide;
iii) detecting the amount of the labeled nucleotide which is incorporated into extended primers for each said location; and
iv) repeating steps ii) and iii) to determine a portion of sequence of each of said colony.

66. The method of claim 65, wherein said labeled nucleotide is a fluorecently-labeled nucleotide, and wherein said detecting involves detecting the fluorescence intensity of said labeled nucleotide.

67. The method of claim 1, wherein said step of determining said set of restriction sequence tags is carried out by a method comprising
B1) linking said restriction fragments in said set of restriction fragments with a first engineered nucleic acid to obtain a set of first nucleic acid fragments, said first engineered nucleic acid comprising a predetermined nucleotide sequence comprising a recognition site of a second restriction enzyme different from said first restriction enzyme;
B2) digesting said first nucleic acid fragments with said second restriction enzyme to obtain a set of second nucleic acid fragments;
B3) linking the ends of said second restriction fragments to produce a set of first circular nucleic acid fragments;
B4) sequencing at least a portion of each of said fourth nucleic acid fragments, thereby determining said set of restriction sequence tags.

68. The method of claim 67, further comprising after said step 3) the steps of
B5) digesting said first circular nucleic acid fragments with a third restriction enzyme to produce a set of third nucleic acid fragments, wherein said third restriction enzyme is different from said first and second restriction enzyms;
B6) modifying the ends generated by said third restriction enzyme to permit ligation; and
B7) linking the ends of said third nucleic acid fragments to produce a set of second circular nucleic acid fragments.

69. The method of claim 67, further comprising repeating said steps B1) through B4) for each of a plurality of different second restriction enzymes.

70. The method of claim 69, further comprising before said step B4) a step of fixing and amplifying nucleic acid fragments in said first circular nucleic acid fragments on a solid surface.

71. The method of claim 70, wherein said step of fixing and amplifying is carried out by generating colonies of said nucleic acid fragments in said first circular nucleic acid fragments on said solid surface, wherein each of said colonies comprises a plurality of immobilized single stranded DNA molecules of one of said nucleic acid fragments in said first circular nucleic acid fragments.

72. The method of claim 71, wherein said colonies are generated by a method comprising
i) linearizing said first circular nucleic acid fragments to generate linearized fragments;
ii) providing a solid surface comprising a plurality of colony primers immobilized on said solid surface at 5′ end, wherein each said colony primer comprises a sequence that is hybridizable to a sequence at the 3′ end of said linearized fragments;
iii) denaturing said linearized fragments to generate single stranded fragments;
iv) annealing said single stranded fragments to said immobilized colony primers;
v) carrying out primer extension reaction using said annealed single stranded fragments as templates to generate immobilized double stranded nucleic acid fragments;
vi) denaturing said immobilized double stranded nucleic acid fragments to generate immobilized single stranded fragments;
vii) annealing said immobilized single stranded fragments to immobilized colony primers;
viii) repeating said steps v) through vii) such that said colonies are generated, each at a particular location on said solid surface.

73. The method of claim 71, wherein said colonies are generated by a method comprising
i) linearizing said first circular nucleic acid fragments to generate linearized fragments;
ii) mixing said linearized fragments with colony primers, wherein each said colony primers comprises a sequence that is hybridizable to a sequence at the 3′ end of said linerarized fragments;
iii) grafting said linearized fragments and colony primers on a solid surface at the 5′ end to generate immobilized linearized fragments and immobilized colony primers;
iv) denaturing said immobilized linearized fragments to generate immobilized single-stranded fragments;
v) annealing said immobilized single stranded fragments to immobilized colony primers to obtain annealed single-stranded fragments;
vi) carrying out primer extension reaction using said annealed single stranded fragments as templates to generate immobilized double stranded nucleic acid fragments;
vii) denaturing said immobilized double stranded nucleic acid fragments to generate immobilized single stranded fragments;
viii) annealing said immobilized single stranded fragments to immobilized colony primers; and
ix) repeating said steps v) through viii) such that said colonies are generated, each at a particular location on said solid surface.

74. The method of claim 71, wherein said colonies are generated by a method comprising
i) linearizing said first circular nucleic acid fragments to generate linearized fragments;
ii) mixing said linearized fragments with colony primers, wherein each said colony primers comprises a sequence that is hybridizable to a sequence at the 3′ end of said linerarized fragments, and wherein the concentration of said colony primers is adjusted such that amplification of grafted linearized fragments can occur;
iii) grafting said linearized fragments and colony primers on a solid surface at the 5′ end to generate immobilized linearized fragments and immobilized colony primers;
iv) applying an amplification solution containing a polymerase and nucleotides to said solid surface such that said colonies are generated isothermally, each at a particularly location on said solid surface.

75. The method of any one of claims 72-74, wherein said sequencing is carried out by a method comprising
i) hybridizing sequencing primers to said colonies;
ii) carrying out primer extension with one labeled nucleotide;
iii) detecting the amount of the labeled nucleotide which is incorporated into extended primers for each said location; and
iv) repeating steps ii) and iii) to determine a portion of sequence of each of said colony.

76. The method of claim 75, wherein said labeled nucleotide is a fluorecently-labeled nucleotide, and wherein said detecting involves detecting the fluorescence intensity of said labeled nucleotide.

77. The method of any one of claims 1, 2, 14, 22, 34, 44, 53, and 67, further comprising in said step A) digesting said set of restriction fragments with a plurality of different first restriction enzymes.

78. The method of any one of claims 1, 2, 14, 22, 34, 44, 53, and 67, wherein each said group consists of restriction sequence tags that are at least 60% homologous.

79. The method of claim 78, wherein each said group consists of restriction sequence tags that are at least 70% homologous.

80. The method of claim 79, wherein each said group consists of restriction sequence tags that are at least 80% homologous.

81. The method of claim 80, wherein each said group consists of restriction sequence tags that are at least 90% homologous.

82. The method of claim 81, wherein each said group consists of restriction sequence tags that are at least 99% homologous.

83. A method for determining genome-wide sequence variations among a plurality of different phenotypes, comprising,
A) determining for each of a population of organisms a set of restriction sequence tags by the method of any one of claims 1, 2, 14, 22, 34, 44, 53, and 67, said population of organisms comprising for each of said plurality of different phenotypes one or more organisms;
B) comparing said sets of restriction sequence tags among organisms of different phenotypes so as to determine one or more sequence variations that associate with different phenotypes.

84. The method of claim 83, further comprising after said step B) a step of mapping said one or more restriction sequence tags to the genomic sequence of said organism so as to identify genomic locations of said one or more restriction sequence tags.

85. The method of any one of claims 45, 55, and 69, wherein said plurality of different second restriction enzymes comprises at least 3 different restriction enzymes.

86. A method for determining genome-wide sequence variations among a plurality of different phenotypes, comprising
A) determining for each of a population of organisms a set of restriction sequence tags by the method of claim 85, said population of organisms comprising for each of said plurality of different phenotypes one or more organisms;
B) comparing said sets of restriction sequence tags among organisms of different phenotypes so as to determine one or more sequence variations that associate with different phenotypes.

87. The method of claim 86, further comprising after said step B) a step of mapping said one or more restriction sequence tags to the genomic sequence of said organism so as to identify genomic locations of said one or more restriction sequence tags.

88. The method of any one of claims 45, 55, and 69, wherein said plurality of different second restriction enzymes comprises at least 10 different restriction enzymes.

89. A method for determining genome-wide sequence variations among a plurality of different phenotypes, comprising
A) determining for each of a population of organisms a set of restriction sequence tags by the method of claim 88, said population of organisms comprising for each of said plurality of different phenotypes one or more organisms;
B) comparing said sets of restriction sequence tags among organisms of different phenotypes so as to determine one or more sequence variations that associate with different phenotypes.

90. The method of claim 89, further comprising after said step B) a step of mapping said one or more restriction sequence tags to the genomic sequence of said organism so as to identify genomic locations of said one or more restriction sequence tags.

91. The method of any one of claims 1, 2, 14, 22, 34, 44, 53, and 67, wherein said one or more individual organisms are humans.

92. The method of any one of claims 1, 2, 14, 22, 34, 44, 53, and 67, wherein each said set of restriction fragments comprises at least 10 different restriction fragments.

93. The method of any one of claims 1, 2, 14, 22, 34, 44, 53, and 67, wherein each said set of restriction fragments comprises at least 100 different restriction fragments.

94. The method of any one of claims 1, 2, 14, 22, 34, 44, 53, and 67, wherein each said set of restriction fragments comprises at least 1000 different restriction fragments.

95. The method of any one of claims 1, 2, 14, 22, 34, 44, 53, and 67, wherein each said set of restriction fragments comprises at least 10,000 different restriction fragments.

96. The method of any one of claims 1, 2, 14, 22, 34, 44, 53, and 67, wherein each said set of restriction fragments comprises at least 100,000 different restriction fragments.

97. The method of any one of claims 1, 2, 14, 22, 34, 44, 53, and 67, wherein each said set of restriction fragments comprises at least 10⁶ different restriction fragments.

98. The method of any one of claims 1, 2, 14, 22, 34, 44, 53, and 67, wherein each said set of restriction fragments comprises at least 10⁷ different restriction fragments.

99. The method of any one of claims 1, 2, 14, 22, 34, 44, 53, and 67, wherein each said set of restriction fragments comprises at least 10⁸ different restriction fragments.

100. The method of claim 1, wherein said step I) is carried out for one individual.

101. The method of claim 1, wherein said step II) of grouping restriction sequence tags further comprises comparing said restriction sequence tags to reference sequences.

102. The method of claim 101, wherein said reference sequences comprise the genomic sequence of the organism.
